SEQUENCE LISTING (23 sequences)

SEQ ID NO: 1 - protein, 377 aa, Salmonella enterica subsp. enterica serovar Typhi Ty2
MKVKVLSLLVPALLVAGAANAAEIYNKDGNKLDLFGKVDGLHYFSDDGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 2 - protein, 363 aa, Salmonella enterica subsp. enterica serovar Typhi CT18
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 3 - protein, 23 aa, Artificial Sequence
  M2e peptide from Human H1N1 e.g. A/USRR/90/77 and A/WSN/33
SLLTEVETPIRNEWGCRCNDSSD

SEQ ID NO: 4 - protein, 23 aa, Artificial Sequence
  M2e peptide
SLLTEVETPIRNEWGCRCNGSSD

SEQ ID NO: 5 - protein, 23 aa, Artificial Sequence
  M2e peptide
SLLTEVETPTKNEWDCRCNDSSD

SEQ ID NO: 6 - protein, 23 aa, Artificial Sequence
  M2e peptide from Equine H3N8 A/equine/Massachussetts/ 213/2003
SLLTEVETPTRNGWECKCSDSSD

SEQ ID NO: 7 - protein, 23 aa, Artificial Sequence
  M2e peptide from H5N1 A/Vietnam/1196/04
SLLTEVETPTRNEWECRCSDSSD

SEQ ID NO: 8 - protein, 24 aa, Artificial Sequence
  M2e peptide from Human H1N1 e.g. A/USRR/90/77 and A/WSN/33
MSLLTEVETPIRNEWGCRCNDSSD

SEQ ID NO: 9 - protein, 24 aa, Artificial Sequence
  M2e peptide from Avian H5N1 A/Vietnam/1196/04
HSLLTEVETPTRNEWECRCSDSSD

SEQ ID NO: 10 - protein, 24 aa, Artificial Sequence
  M2e peptide from H3N8, Horse-Dog A/equine/Massachussetts/ 213/2003
MSLLTEVETPTRNGWECKCSDSSD

SEQ ID NO: 11 - protein, 24 aa, Artificial Sequence
  M2e peptide from H9N2, A/chicken/Osaka/aq69/2001
MSLLTEVETPTRNGWGCRCSDSSD

SEQ ID NO: 12 - protein, 24 aa, Artificial Sequence
  M2e peptide from Mutant H1N1 I/T
MSLLTEVETPTRNEWGCRCSDSSD

SEQ ID NO: 13 - protein, 8 aa, Artificial Sequence
  M2e conserved epitope
EVETPIRN

SEQ ID NO: 14 - protein, 8 aa, Artificial Sequence
  Variant of M2e conserved epitope
EVETLTRN

SEQ ID NO: 15 - protein, 8 aa, Artificial Sequence
  Variant of M2e conserved epitope
EVETPIRS

SEQ ID NO: 16 - protein, 8 aa, Artificial Sequence
  Variant of M2e conserved epitope
EVETPTRN

SEQ ID NO: 17 - protein, 8 aa, Artificial Sequence
  Variant of M2e conserved epitope
EVETPTKN

SEQ ID NO: 18 - protein, 8 aa, Artificial Sequence
  Variant of M2e conserved epitope
EVDTLTRN

SEQ ID NO: 19 - protein, 8 aa, Artificial Sequence
  Variant of M2e conserved epitope
EVETPIRK

SEQ ID NO: 20 - protein, 8 aa, Artificial Sequence
  Variant of M2e conserved epitope
EVETLTKN

SEQ ID NO: 21 - protein, 363 aa, Salmonella typhi
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGAGGSADLAKYIQAGATYYFNKNMNVWVDYRFNLLDENDYSSSYVGTDDQAAVGITYQF

SEQ ID NO: 22 - DNA, 1137 bp, Salmonella typhi
atgaaagttaaagtactgtccctcctggtaccagctctgctggtggcgggcgcagcgaatgcggctgaaatttataataaagacggcaacaaattagacctgtttggtaaagttgatggcctgcactacttctctgacgacaaaggcagcgacggcgaccagacctacatgcgtatcggcttcaaaggcgaaacgcaggttaacgatcagctgaccggttatggccagtgggaatatcagattcagggcaaccagactgaaggcagcaacgactcctggacgcgtgtggcgtttgcgggtctgaaattcgctgacgcaggttccttcgattatggtcgtaactacggcgtaacctatgacgtgacctcctggaccgacgttctgccggagttcggcggcgacacctacggcgctgacaactttatgcagcagcgtggtaacggctatgctacctaccgtaacaccgacttcttcggtctggtggatggtctggacttcgcgttgcagtatcagggcaaaaacggcagcgtgagcggtgaaaacaccaacggtcgcagcctgctgaaccagaacggcgacggttacggcggatcgctgacttatgcaatcggcgaaggcttctctgtcggtggcgctatcaccacgtctaaacgtactgccgatcagaacaacaccgctaacgctcgcctgtatggtaacggcgatcgcgccacggtttacaccggcggcctgaaatacgatgcgaacaacatctatctggcagcgcagtattctcagacctataacgcaacccgttttggtacctctaacggcagcaacccgtccacctcttacggttttgccaacaaagcgcagaactttgaagtggttgctcagtaccagttcgactttggtctgcgtccgtctgtggcttacctgcagtctaaaggtaaggacatcagcaacggctacggcgccagctatggcgaccaggacatcgtaaaatacgttgatgtcggcgcgacttactacttcaacaaaaacatgtccacctatgttgattacaaaatcaacctgctggataaaaacgactttacccgcgatgcgggcatcaacaccgacgacatcgtagcgctgggtctggtttaccagttctaa

SEQ ID NO: 23 - DNA, 1092 bp, Salmonella typhi
atgatgaagcgcaaaatcctggcagcggtgatccctgccctgctggctgctgcaaccgcaaacgcagcagaaatttataataaagatggtaataagctggatctgtacggtaaagccgtgggtcgtcacgtatggacaacgaccggcgatagtaaaaatgccgaccagacttatgcccagattggttttaaaggggaaacgcagattaacaccgatctgaccggtttcggtcagtgggaataccgtactaaagcagaccgcgctgaaggcgaacagcagaactcgaatctggtacgtctggctttcgcgggtttgaaatacgcggaagtgggttcaatcgattatggtcgtaactacggtatcgtttacgatgttgaatcctataccgatatggccccctacttctccggcgaaacctggggcggcgcctataccgataactacatgaccagccgtgctggcggtttgttgacctaccgtaactctgacttctttggtctggtggacggtctctctttcggtatccagtatcagggtaaaaatcaggacaaccacagcattaactctcagaatggcgatggcgtaggttacaccatggcatatgagttcgacggctttggcgtcaccgcagcgtacagcaacagcaagcgtactaacgatcagcaggatcgcgatggtaatggcgatcgcgcagaatcgtgggccgttggcgcgaaatatgatgcaaacaacgtctacctggctgccgtatatgctgaaacccgcaatatgagcattgttgaaaatacggttaccgataccgtggaaatggcaaacaaaacgcagaacctggaagtggtcgctcagtaccagtttgacttcggcctgcgtccggcaatctcgtatgtgcagagtaaaggtaagcagttgaacggcgccgacggctcggccgatctggcgaaatatattcaggcgggcgcgacttactacttcaacaaaaacatgaacgtatgggttgactaccgtttcaacctgctggacgaaaacgactacagctccagctacgttggcaccgacgatcaggcggctgtcggtattacttaccagttctga

(19) United States
(12) Patent Application Publication
(10) Pub. No.: US 2011/0189231 A1
(43) Pub. Date: Aug. 4, 2011

(54) Compositions Comprising Salmonella Porins and Uses Thereof as Adjuvants and Vaccines

(76) Inventors: Denis Leclerc, Fossambault-sur-le-Lac (CA); Constantino Roberto Lopez-Macias, III, Mexico City (MX)

(21) Appl. No.: 12/999,989

(22) PCT Filed: Jun. 17, 2009
(86) PCT No.: PCT/CA2009/000816

§ 371 Date: Apr. 22, 2011

Related U.S. Application Data
(60) Provisional application No. 61/073,118, filed on Jun. 17, 2008.

(51) Int. Cl. A61K 39/145; A61K 39/39; A61P 37/04; A61P 31/16

(52) U.S. Cl. 424/209.1; 424/258.1

(57) ABSTRACT

Adjuvants comprising OmpC porin, OmpF porin, or a combination thereof, are provided. The adjuvants can be administered to a subject in combination with antigenic material in order to potentiate the immunogenic effect of the antigenic material. Also provided are products comprising antigenic material in combination with OmpC and/or OmpF, including products comprising a pre-formulated vaccine in combination with OmpC and/or OmpF. Further provided is the use of OmpC and/or OmpF to improve the effect of a pre-formulated vaccine.

### FIELD OF THE INVENTION

The present invention relates to the field of vaccine formulations and adjuvants and, in particular, to vaccines and adjuvants based on the OmpC and OmpF porins from Salmonella spp.

### BACKGROUND OF THE INVENTION

Adjuvants are frequently used in vaccine preparations in order to enhance the ability of antigens to induce protective immune responses in a host. The most commonly utilised adjuvants in injectable human vaccines are alum-based adjuvants.

Other compounds and molecules have been investigated for their potential to be used as adjuvants. For example, bacterial enterotoxins (such as mutated cholera toxin and heat-labile toxins) have shown promise as nasally delivered mucosal adjuvants, however, development of these adjuvants has been hindered due to their ability to be transported to, and cause inflammation in the olfactory bulb region of the CNS of rodents.

Lipopolysaccharides (LPS) from gram negative bacteria are known to be potent adjuvants, but the use of LPS in humans has been restricted due to the associated endotoxicity mediated by the lipid A portion of the molecule. Chemical modification of the lipid A region of LPS was shown to substantially detoxify lipid A while maintaining certain adjuvant properties (Qureshi et al., J. Biol Chem (1982) 257:11808-15).

The ability of bacterial outer membrane proteins (OMPs) to enhance the immune response to poorly immunogenic substances has been described. The outer membrane protein complex from Neisseria meningitis has been used as an adjuvant for low immunogenic antigens (U.S. patent application Ser. No. 10/003,463 (2002/0136735)) including the melanoma antigen GD3 (Livingston et al., Vaccine (1993) 11:1199-1204), capsular polysaccharide from Haemophilus influenzae (Donnelly et al., J. Immunol. (1990) 145:3071-3079; Latz et al., J. Immunol. (2004) 172:2431-2438), and recombinant Pfs25H, a malarial vaccine candidate (Wu et al., PNAS (2006) 103:18243-18248). PedvaxHIB® (Merck & Co., Inc.) is a commercially available conjugate vaccine against invasive Haemophilus influenzae type b disease that contains the outer membrane complex of Neiserria meningitis serogroup B. In addition, the PorB porin from commensal Neisseria lactamica has been shown to induce Th1 and Th2 immune responses to ovalbumin in mice and is a potential immune adjuvant (Liu, et al., Vaccine (2008) 26:786-96).

Dalseg et al. (in Vaccines 96 pp. 177-182 (Cold Spring Harbor Laboratory Press, 1996)) report the use of meningococcal outer membrane vesicles (OMVs) as a mucosal adjuvant for inactivated whole influenza virus.

International Patent Application No. PCT/US02/07108 (WO 02/072012) and U.S. patent application Ser. No. 10/094,424 (2003/0044425) describe an adjuvant complex composed of bacterial outer membrane protein proteosomes, and specifically Neisseria meningitis outer membrane protein proteosomes, complexed to bacterial LPS. The LPS is derived from Shigella, Plesiomonas, Escherichia or Salmonella species.

International Patent Application No. PCT/FR01/03596 (WO 02/40518) describes the use of a periplasmic domain of an enterobacterial OMP, and specifically a Klebsiella pneumonae OMP, as a carrier or adjuvant in vaccine preparations.

Porins from Salmonella typhimurium have been shown to be potent polyclonal activators for murine B lymphocytes (Vordermeier et al., Immunobiology (1987) 175:245-251). These porins and small fragments of the porins have also been shown to be potent mitogens for human peripheral blood lymphocytes (Vordermeier et al., Immunol. Lett. (1987) 15:121-126). Purified porins from S. typhimurium have also been shown to stimulate an immune response in mice sufficient to provide protection against a subsequent challenge with S. typhimurium (Galdiero et al., Immunology (1998) 94:5-13).

Porins from Salmonella typhi have been shown to elicit a host immune response and provide protection of the host against S. typhi infection, and as such have been studied as candidates for a typhoid fever vaccine (Salazar-Gonzalez et al., Immunol. Lett. (2004) 93:115-122). A preparation comprising OmpC and OmpF porins from S. typhi has been shown to trigger a strong long-lasting immunoglobulin G (IgG) production in BALB/c mice in the absence of exogenous adjuvant. Evaluation of the individual contribution of each porin to this long-lasting antibody response suggested that the main protein responsible for the antibody-mediated memory response was OmpC. The response was shown to be highly specific as the anti-porin sera did not cross-react with S. typhimurium despite the high homology of the porins from these two Salmonella species (Secundino et al., Immunology (2006) 117:59-70).

Vaccines have been developed that rely on the generation of a humoral or antibody response which targets surface antigens on a pathogen. Recently, efforts have been directed towards the development of adjuvants and vaccines that induce a protective cellular immune response mediated by CTL (cytotoxic T lymphocyte) cells. Such adjuvants and vaccines could provide advantages over those that generate mainly a humoral response, particularly when the target antigen is one that constantly mutates, such as for example, the influenza virus.

Existing influenza vaccines contain three killed or attenuated virus strains—one A (H3N2) virus, one A (H1N1) virus, and one B virus, and the viruses in the vaccine change each year. Hemagglutinin (HA) and neuraminidase (NA), two accessible large glycoproteins at the surface of the virus, are the major target of the immune response during infection which induces their drift and their shift. The selection pressure of the immune system on these surface glycoproteins favours the emergence of mutated virus that propagates efficiently and causes new epidemics. The newly emerged strain is selected to be a component of the next generation of the vaccine, which may come on the market 6-8 months later. During this time, however, circulating virus has time to evolve resulting in partial efficiency of the vaccine, once it is administered. Moreover, the reassortment of the virus in pig and bird reservoirs further complicates the cycle and can be the source of pandemics. For example, during the 20th century, the emergence of several new influenza A virus subtypes caused three pandemics: “Spanish flu” [A (H1N1)] (1918), “Asian flu” [A (H2N2)] (1957) and the “Hong Kong flu” [A (H3N2)] (1968), all of which spread around the world within a year of being detected.

According to existing vaccine paradigm, it is believed that vaccination would not be effective for preventing a pandemic because it targets individual viral strains and not the entire influenza virus class (Shoham, D. (2006) Virus Genes 33:127-132.). A live attenuated nasal influenza vaccine (FluMist; MedImmune, Inc.) has been produced and can provide a certain level of cross protection to other strains of influenza through induction of a cytotoxic T lymphocyte (CTL) response toward highly conserved protein found inside the virus particle (Kaiser et al., (2006) Int Rev Immunol. 25; 99-123). However, there is a risk that this vaccine strain can revert to a dangerous form which could create a new pandemic strain (Kaiser (2006), supra).

One approach to developing a universal influenza vaccine is to use conserved internal proteins such as the matrix protein (M1) or the nucleocapsid (NP) to elicit immunity based on CTL rather than neutralizing antibodies to HA and NA (Thomas et al., (2006), Emerg Infect Dis. 12:48-54). CTLs would eliminate the infected cells by specific recognition of influenza peptides loaded on the MHC class I complex. The MHC class I complex, located at the surface of the infected cells, efficiently presents peptides derived from highly conserved proteins like M1 and NP. The target proteins must, however, be associated with an adjuvant or a delivery system that will bring the antigen to the pathway of degradation and presentation of peptides on MHC class I complex of the antigen presentation cells (APC). Several delivery systems such as the adenovirus vectors (Bangari and Mittal, (2006) Curr Gene Ther. 6; 215-226) and DNA vaccines (Laddy and Weiner, (2006) Int Rev Immunol. 25; 99-123) are aimed at developing a CTL response to conserved epitopes. However, adenovirus vectors can be neutralized by naturally resident antibodies that inhibit their entry to APC and DNA vaccines are not immunogenic in large animals. Although promising, these systems are in general not sufficiently immunogenic.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide compositions comprising Salmonella porins and uses thereof as adjuvants and vaccines. In accordance with one aspect of the invention, there is provided a composition comprising OmpC porin, or OmpF porin, or a combination thereof, and a physiologically acceptable carrier or diluent, for use as an adjuvant to potentiate an immune response against antigenic material in a subject in need thereof.

In one embodiment of the invention, there is provided a composition comprising OmpC porin, or OmpF porin, or a combination thereof, and a physiologically acceptable carrier or diluent, for use as an adjuvant to potentiate an immune response against antigenic material in a subject in need thereof, wherein the antigenic material is derived from one or more strains of influenza virus.

In accordance with another aspect of the invention, there is provided a composition comprising OmpC porin, or OmpF porin, or a combination thereof, and a physiologically acceptable carrier or diluent, for use to improve the efficacy of a vaccine whereby a subject treated with said composition and said vaccine shows an improved immune response over a subject treated with said vaccine alone.

In accordance with another aspect of the invention, there is provided a use of OmpC porin, or OmpF porin, or a combination thereof, in the manufacture of a medicament for potentiating an immune response against antigenic material in a subject in need thereof.

In accordance with another aspect of the invention, there is provided a use of OmpC porin, or OmpF porin, or a combination thereof, in the manufacture of a medicament for improving the efficacy of a vaccine whereby a subject treated with said composition and said vaccine shows an improved immune response over a subject treated with said vaccine alone.

In accordance with another aspect of the invention, there is provided a method of potentiating an immune response in a subject, said method comprising administering to said subject an effective amount of a composition comprising OmpC porin, or OmpF porin, or a combination thereof, and antigenic material.

In accordance with another aspect of the invention, there is provided a method of improving the efficacy of a vaccine comprising administering to a subject said influenza vaccine and a composition comprising OmpC porin, or OmpF porin, or a combination thereof, whereby the subject treated with said influenza vaccine and said composition shows an improved immune response over a subject treated with said influenza vaccine alone.

In accordance with another aspect of the invention, there is provided a product comprising OmpC porin, or OmpF porin, or a combination thereof, and antigenic material, wherein said OmpC porin, or OmpF porin, or combination thereof, is capable of potentiating an immune response to said antigenic material in a subject.

### BRIEF DESCRIPTION OF THE DRAWINGS

These and other features of the invention will become more apparent in the following detailed description in which reference is made to the appended drawings.

FIG. 1 presents (A) the amino acid sequence (SEQ ID NO:1) of the OmpC precursor from Salmonella enterica subsp. enterica serovar Typhi Ty2 (GenBank Accession No. P0A264), and (B) a nucleotide sequence (SEQ ID NO:22) that encodes the amino acid sequence shown in (A).

FIG. 2 presents (A) the amino acid sequence (SEQ ID NO:2) of the OmpF precursor protein from Salmonella enterica subsp. enterica serovar Typhi CT18 (GenBank Accession No. CAD05399), and (B) a nucleotide sequence (SEQ ID NO:23) that encodes the amino acid sequence shown in (A).

FIG. 3 presents the amino acid sequence (SEQ ID NO:21) of the OmpF protein from Salmonella typhi (GenBank Accession No. CAA61905.1).

FIG. 4 presents an SDS-PAGE gel analysis of OmpC and OmpF porins purified from S. typhi strains. Lane 1, molecular weight standards; lane 2, 1.0 μg porins purified from wild type S. typhi ATCC 9993; lane 3, 1.0 μg OmpC purified from S. typhi STYF302; lane 4, 1.0 μg OmpF purified from S. typhi STYC171. Molecular weight standards are indicated on the left.

FIG. 5 shows the results of FACS analysis of the amounts of immune cell populations recruited at the immunization site when BALB/c mice were immunized i.p. with 20 μg of S. typhi porins, (A) Plasma cells (CD138+ IgMlow) (B) Dendritic cells (CD11c+) (C) B2 cells (CD21low/CD23low) and (D) B1b cells (B220 low/CD5−/CD21−/CD23−). Each density plot is representative of 3 experiments.

FIG. 6 presents the results of flow cytometry analysis of the ability of S. typhi porins to up-regulate the expression of co-stimulatory molecules and activation markers in antigen presenting cells (APC): (A) Dendritic cells or (B) Bone Marrow Derived Macrophages (BMDM) were stimulated with 1 μg/mL of porins for 24 hours (DCs) and 48 hours (BMDM), then cells were stained with anti-CD80 FITC, anti-CD86 FITC, anti-CD40 FITC, anti-CD69 FITC or anti-MHCII-FITC antibodies.

FIG. 7 demonstrates that S. typhi porins induce signal through TLR-2 and TLR-4 and induce the production of pro- and anti-inflammatory cytokines on DC: HEK 293 cells transfected with plasmids encoding for TLR-4/MD2 (A) or TLR-2 (B) were stimulated with porins 1 μg/mL, proteinase K degraded porins (porins K), LPS, Zymosan or a porin purification preparation in which porins were depleted by flocculation/filtration (porins SP). TLR signaling was measured by IL-8 secretion using ELISA. (C) Dendritic cells were stimulated with 1 μg/mL of OmpC or OmpF, the supernatants were collected at 6, 12 or 24 hours and the presence of IL-6, TNF-a and IL-10 was analyzed by ELISA.

FIG. 8 shows the IgG titers against the model antigens hen egg lysozyme (HEL) and ovalbumin (OVA) in BALB/c mice—Groups of 3 BALB/c mice were co-immunized with (A) 10 μg of a S. typhi porin preparation, OmpC or OmpF and 1 mg of HEL or (B) 10 μg of a S. typhi porin preparation, OmpC or OmpF and 2 mg of OVA. 5 μg of LPS or Freund's Complete Adjuvant (FCA) were used as controls. Blood samples were obtained on the indicated days and the IgG titer was analysed by ELISA.

FIG. 9 shows (A) the weight and (B) the bacterial numbers per spleen for C57/BL6 mice (5 per group) immunized i.p. on day 0 with 20 μg of S. typhi porins (typhi por), 20 μg of S. typhimurium porins (typhimu por) or both (typhi+typhimu por). At day 35, the mice were infected with 10⁴ CFU of PhoP⁻ S. typhimurium. Measurements were made five days after infection (day 40).

FIG. 10 shows total IgG titers against the haemagglutinin proteins of the Fluviral® vaccine as measured by ELISA for BALB/c mice that received one subcutaneous injection of Fluviral® vaccine (3 μg, which is equivalent to one-fifth the human dose) or of Fluviral® adjuvanted with either 3 μg or 30 μg of purified OmpC. The red line represents the normal baseline for pre-immunised mice. *** p<0.001 vs. Fluviral®.

FIG. 11 shows (A) IgG2a titers and (B) IgG1 titers against the haemagglutinin proteins of the Fluviral® vaccine as measured by ELISA for BALB/c mice that received one subcutaneous injection of Fluviral® vaccine (3 μg, which is equivalent to one-fifth the human dose) or of Fluviral® adjuvanted with either 3 μg or 30 μg of purified OmpC. The red line represents the normal baseline for pre-immunised mice. *** p<0.001 vs. Fluviral®.

FIG. 12 shows (A) total IgG titers, and (B) IgG1 titers against the influenza virus NP protein as measured by ELISA for BALB/c mice that received one subcutaneous injection of Fluviral® vaccine (3 μg, which is equivalent to one-fifth the human dose) or of Fluviral® adjuvanted with 3 μg or 30 μg of purified OmpC. The red line represents the normal baseline for pre-immunised mice.

FIG. 13 shows IgG2a titers against the influenza virus NP protein as measured by ELISA for BALB/c mice treated as described for FIG. 12. The red line represents the normal baseline for pre-immunised mice. * p<0.05 vs. Fluviral® and ** p<0.01 vs. Fluviral®.

FIG. 14 presents the results of a challenge of vaccinated mice with 4,000 pfu of the heterologous influenza strain WSN/33. BALB/c mice were vaccinated with one subcutaneous injection of Fluviral® vaccine (3 μg, which is equivalent to one-fifth the human dose) or of Fluviral® adjuvanted with 30 μg of purified OmpC: (A) shows the change in body weight of the mice as measured daily for 14 days after challenge, (B) shows the symptoms presented by the mice scored according to Table 4 on a daily basis for 14 days after challenge, and (C) shows the survival rate of the mice.

FIG. 15 presents a comparison of the humoral response induced in BALB/c mice after immunisation with 3 μg (equivalent to one-fifth of the human dose) of the commercial vaccine Fluviral® alone or with 30 μg of purified OmpC. Total IgG (A), IgG1 (B) and IgG2a (C) to Fluviral® proteins as measured 2 months after immunisation; total IgG (D), IgG1 (E) and IgG2a (F) to NP as measured 2 months after immunisation, and total IgG (G), and IgG2a (H) against Fluviral® proteins 10 months after immunization. (* p>0.05, ** p>0.01 and *** p>0.0001).

FIG. 16 presents the results of a challenge vaccinated of mice with 100 pfu of the heterologous influenza strain WSN/33. Mice were vaccinated with Fluviral® alone, or in combination with 30 μg of OmpC (“Flu+OmpC”) and challenged 10 months after vaccination. (A) shows the change in body weight of surviving mice as measured daily for 13 days after challenge, (B) shows the survival rate of the mice, and (C) shows the symptoms presented by the mice scored according to Table 4 on a daily basis for 13 days after challenge.

FIG. 17 shows (A) IgM titers, and (B) IgG titers against the Mycobacterium tuberculosis p38 protein as measured by ELISA for BALB/c mice that received one intraperitoneal injection of p38 protein (10 μg) or of p38 protein adjuvanted with 10 μg of purified OmpC.

### DETAILED DESCRIPTION OF THE INVENTION

The present invention relates to the ability of the OmpC and OmpF porins from Salmonella enterica subsp. enterica serovar Typhi (“Salmonella typhi”) to function as adjuvants to potentiate the immune response to antigenic material in a subject. In one aspect, therefore, the invention provides for the use of an OmpC porin, an OmpF porin, or a combination thereof, as an adjuvant. In another aspect, the invention provides for immunogenic compositions comprising OmpC and/or OmpF suitable for use as an adjuvant. The porins may be the S. typhi wild-type porin proteins or they may be porin proteins having an amino acid sequence substantially identical to the S. typhi wild-type porin protein sequence, including substantially similar porin proteins found in other Salmonella species, as described in more detail below. The porin proteins may be provided as purified proteins, partially purified proteins or as a crude cellular extract. The adjuvant can be used to potentiate the immunogenic effect of known antigens by administering the adjuvant to a subject in combination with antigenic material.

The invention further provides for immunogenic compositions and combination products comprising antigenic material in combination with OmpC and/or OmpF. The antigenic material can be purified or partially purified and, in certain embodiments, can be provided in the form of a known vaccine, for example a commercially available vaccine. In accordance with this aspect of the invention, the immunogenic compositions may be provided as a single formulation comprising the porin(s) and the antigenic material. Combination products generally comprise two or more separate formulations, one comprising OmpC and/or OmpF and optionally antigenic material, and the other(s) comprising antigenic material alone. The formulations comprised by the combination product can be administered to the subject separately, or concomitantly, for example by combining the formulations prior to administration. In certain embodiments, the formulation comprising antigenic material is a commercially available vaccine.

In one embodiment of the invention, S. typhi OmpC and/or OmpF is used to potentiate the immunogenic effect of a vaccine comprising multiple epitopes, for example, a whole cell vaccine, or vaccine comprising a complex mixture of proteins and/or other cellular components. In another embodiment of the invention, OmpC and/or OmpF is used to potentiate immunoprotective effects against diseases or disorders that require the participation of antibody and T cell immune responses in order to be effective. For example, immunoprotective effects against influenza, hepatitis B, hepatitis C, human immunodeficiency virus (HIV), human T-lymphotropic virus (HTLV), Dengue virus, malaria, and systemic bacterial infections (such as those that occur in typhoid fever, Leishmania major infection or Mycobacterium tuberculosis infection), which are most effective when a cellular response is induced, as well as immunotherapeutic treatment of cancer.

S. typhi OmpC alone or in combination with S. typhi OmpF is known in the art to be capable of providing protection against infection by S. typhi, the agent that causes typhoid fever. Combination of OmpC and optionally OmpF with antigenic material from a different disease-causing agent, therefore, will provide a multivalent vaccine or combination product in which the OmpC/OmpF component provides protection against S. typhi and also adjuvants the other antigen(s) in the vaccine or combination product to provide protection against other disease(s). Additionally, due to the high homology between OmpC and OmpF of Salmonella spp. and the corresponding porins in other enterobacteria, in some embodiments, Salmonella spp. OmpC and/or OmpF can also be used to provide protective effects against infection with other enterobacteria. Accordingly, one embodiment of the invention provides for multivalent vaccines or combination products that induce protective immune responses in a subject against two or more diseases.

In a specific embodiment of the invention, S. typhi OmpC and/or OmpF is used to potentiate an immune response to antigenic material from the influenza virus. In one embodiment of the invention, OmpC and/or OmpF is used to potentiate an immune response to antigenic material from the influenza virus, in which the immune response provides protection against multiple influenza strains. In another embodiment of the invention, OmpC and/or OmpF is used to adjuvant an influenza vaccine and produce an immune response that provides protection against multiple influenza strains, including strains against which the vaccine alone does not provide protection (referred to herein as “heterologous influenza strains”). In a further embodiment, vaccine compositions comprising OmpC and/or OmpF and antigenic material from the influenza virus and combination products comprising OmpC and/or OmpF and an influenza vaccine are provided. In another embodiment of the invention, there is provided a multivalent vaccine preparation or combination product comprising S. typhi OmpC and/or OmpF and antigenic material from the influenza virus that provides protection against both typhoid fever and influenza.

One embodiment of the invention also provides for the use of OmpC and/or OmpF to adjuvant DNA vaccines. In accordance with this embodiment, the OmpC or OmpF gene is cloned into an appropriate eukaryotic expression vector behind a suitable promoter to allow expression of OmpC/OmpF in the target host cell. OmpC and/or OmpF can be co-expressed with the chosen antigen either by cloning the genes in tandem, expressing the porin and the antigen as a fusion protein in which the antigen is inserted into an external loop of the porin, or by co-transfecting the host cell with two expression vectors, one expressing the porin and the other expressing the antigen.

### DEFINITIONS

Unless defined otherwise, all technical and scientific terms used herein have the same meaning as commonly understood by one of ordinary skill in the art to which this invention belongs.

As used herein, the term “about” refers to approximately a +/−10% variation from a given value. It is to be understood that such a variation is always included in any given value provided herein, whether or not it is specifically referred to.

The terms “antigenic material” or “antigen” as used herein refer to a molecule, molecules, a portion or portions of a molecule, or a combination of molecules, up to and including whole cells of prokaryotic or eukaryotic origin and viruses, which are capable of inducing an immune response in a subject alone or in combination with an adjuvant. The antigen may comprise a single epitope or may comprise a plurality of epitopes. The term thus encompasses peptides, carbohydrates, proteins, nucleic acids, lipids, and combinations thereof, as well as various microorganisms, in whole or in part, including viruses, bacteria and parasites. Haptens are also considered to be encompassed by the terms “antigenic material” and “antigen” as used herein.

The term “adjuvant,” as used herein, refers to an agent that potentiates and/or promotes an immune response in an animal to antigenic material. An adjuvant may or may not itself elicit an immune response.

The term “potentiate” and grammatical variations thereof as used herein with respect to an adjuvant's ability to potentiate an immune response to antigenic material means to make effective or active or more effective or more active.

The term “immune response,” as used herein, refers to an alteration in the reactivity of the immune system of an animal to antigenic material and may involve antibody production, innate immunity activation, induction of cell-mediated immunity, complement activation, or development of immunological tolerance, or a combination thereof.

The terms “effective immunoprotective response” and “immunoprotection,” as used herein, mean an immune response that is directed against antigenic material so as to protect against disease and/or infection caused by the antigenic material or source of the antigenic material in a subject. For purposes of the present invention, protection against disease and/or infection includes not only the absolute prevention of the disease or infection, but also any detectable reduction in the degree or rate of disease or infection, or any detectable reduction in the severity of the disease or any symptom or condition resulting from infection in the treated subject as compared to an untreated infected or diseased subject. An effective immunoprotective response can be induced in a subject that was not previously suffering from the disease, was not previously infected with the pathogen and/or does not have the disease or infection at the time of treatment. An effective immunoprotective response can also be induced in a subject already suffering from the disease or infection at the time of treatment. Immunoprotection can be the result of one or more mechanisms, including humoral and/or cellular immune responses.

“Naturally-occurring,” as used herein, as applied to an object, refers to the fact that an object can be found in nature. For example, an organism (including a virus), or a polypeptide or polynucleotide sequence that is present in an organism that can be isolated from a source in nature and which has not been intentionally modified by man in the laboratory is naturally-occurring.

The terms “polypeptide” or “peptide” as used herein is intended to mean a molecule in which there is at least four amino acids linked by peptide bonds.

The term “vaccine,” as used herein, refers to a material capable of producing an effective immunoprotective response in a subject.

The terms “immunization” and “vaccination” are used interchangeably herein to refer to the administration of a vaccine to a subject for the purposes of raising an immune response and can have a prophylactic effect, or a therapeutic effect, or a combination thereof. Immunization can be accomplished using various methods depending on the subject to be treated including, but not limited to, intraperitoneal injection (i.p.), intravenous injection (i.v.), intramuscular injection (i.m.), oral administration, intranasal administration, spray administration, topical administration on skin or mucosal surfaces, and immersion.

The term “multivalent” as used herein with reference to a vaccine preparation is intended to mean a vaccine preparation that contains antigenic material from two or more disease-causing agents and which, when administered to a subject, provides protection against these two or more disease-causing agents. The term thus encompasses bivalent, trivalent and higher valency vaccine preparations.

As used herein, the terms “treat,” “treated,” or “treating” when used with respect to a disease or infection refers to a treatment which increases the resistance of a subject to the disease or to infection (i.e. decreases the likelihood that the subject will contract the disease or become infected) as well as a treatment after the subject has contracted the disease or become infected in order to fight a disease or infection (for example, reduce, eliminate, ameliorate or stabilise a disease or infection).

In the context of the present invention, administration of OmpC and/or OmpF “in combination with” antigenic material, is intended to include simultaneous (concurrent) administration and consecutive administration. Consecutive administration is intended to encompass administration to the subject of OmpC and/or OmpF and subsequently the antigenic material, as well as administration of the antigenic material and subsequently OmpC and/or OmpF. When OmpC and/or OmpF and the antigenic material are administered consecutively, the time interval between administration of OmpC and/or OmpF and administration of the antigenic material may be in the range of a few minutes to a few days.

The term “subject” or “patient” as used herein refers to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep and other hoofed animals, dogs, cats, chickens, ducks, non-human primates, guinea pigs, rabbits, ferrets, rats, hamsters and mice.

The term “substantially identical,” as used herein in relation to a nucleic acid or amino acid sequence indicates that, when optimally aligned, for example using the methods described below, the nucleic acid or amino acid sequence shares 70% or greater, 75% or greater, 80% or greater, 85% or greater, 90% or greater, 95% or greater, 96% or greater, 97% or greater, 98% or greater, or 99% or greater sequence identity with a defined second nucleic acid or amino acid sequence (or “reference sequence”). In one embodiment of the invention, substantially identical sequences share 90% or greater sequence identity. In another embodiment of the invention, substantially identical sequences share 95% or greater sequence identity. “Substantial identity” may be used to refer to various types and lengths of sequence, such as full-length sequence, mature (or “processed” sequences), functional domains, coding and/or regulatory sequences, promoters, and genomic sequences. In one embodiment of the invention, substantial identity is established using full-length sequences. In another embodiment, substantial identity is established using mature sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The terms “corresponding to” or “corresponds to” as used herein with reference to a nucleic acid sequence (or polynucleotide) indicate that the nucleic acid sequence is identical to all or a portion of a reference nucleic acid sequence. In contradistinction, the term “complementary to” is used herein to indicate that the nucleic acid sequence is identical to all or a portion of the complementary strand of a reference nucleic acid sequence. For illustration, the nucleic acid sequence “TATAC” corresponds to a reference sequence “TATAC” and is complementary to a reference sequence “GTATA.”

Immunogenic Compositions and Combination Products Comprising Salmonella TYPHI OmpC and/or OmpF

The immunogenic compositions and combination products (referred to herein collectively as “products”) in accordance with the present invention comprise an OmpC porin, an OmpF porin, or a combination thereof. When the product is a combination product, it also comprises antigenic material. When the product is an immunogenic composition, it may optionally comprise antigenic material. The antigenic material can be purified or partially purified, for example, one or more purified or partially purified antigens, or it can be provided in the form of a known vaccine. The products may further optionally comprise a suitable carrier, excipient or the like, and/or other standard components of pharmaceutical compositions that improve the stability, palatability, pharmacokinetics, bioavailability or the like, of the product.

The porin (OmpC and/or OmpF) comprised by the product can be the S. typhi wild-type porin proteins or they may be porin proteins having an amino acid sequence substantially identical to the S. typhi wild-type porin protein sequence, including substantially similar porin proteins found in other Salmonella species. In one embodiment, the products comprise the OmpC porin from Salmonella enterica subsp. enterica serovar Typhi (“Salmonella typhi”), the OmpF porin from Salmonella typhi, or a combination thereof.

The porins can be purified proteins, partially purified proteins or crude extracts. As such, the porin preparation may comprise other cellular components including additional outer membrane proteins, or it may be substantially free of other cellular components. In one embodiment, the porin preparation comprised by the product comprises OmpC and OmpF. In another embodiment, the porin preparation comprised by the product comprises OmpC and optionally OmpF. In another embodiment, the porin preparation comprised by the product comprises OmpC but is substantially free of other cellular components. In a further embodiment, the porin preparation comprised by the product comprises OmpC and OmpF, but is substantially free of other cellular components, such as lipopolysaccharides (LPS).

The products of the invention also include immunogenic compositions and combination products in which the porin and optional antigen(s) are provided in the form of DNA, which expresses the encoded porin and optional antigen(s) upon transfection into the target host cell. In accordance with this aspect of the invention, the product may comprise an expression vector comprising the porin gene behind a suitable promoter alone or combined with an existing DNA vaccine, a single expression vector comprising the porin gene and antigen-encoding sequence cloned in tandem, an expression vector comprising a sequence encoding a fusion protein in which the antigen is inserted into an external loop of the porin, or two or more expression vectors—one comprising a sequence encoding the porin and the other(s) comprising a sequence encoding an antigen.

### OmpC Porin

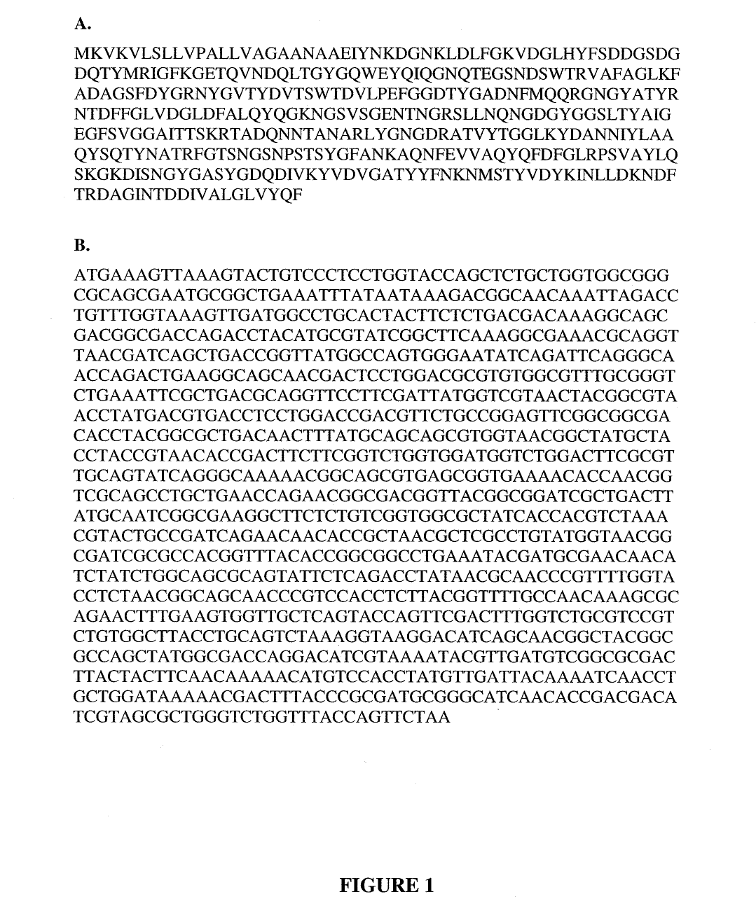

The sequences of OmpC porins from various S. typhi strains are known in the art and are readily accessible, for example, from GenBank database maintained by the National Center for Biotechnology Information (NCBI). For example, GenBank Accession No. P0A264 (SEQ ID NO:1; also shown in FIG. 1A), GenBank Accession No. AAO68302.1 and GenBank Accession No. NP_{—}804453: OmpC (S. enterica subsp. enterica serovar Typhi Ty2); and GenBank Accession No. CAD07499.1 and GenBank Accession No. NP_{—}456812.1: OmpC (S. enterica subsp. enterica serovar Typhi strain CT18).

The OmpC porin for use in the products according to the invention can be obtained from Salmonella typhi by standard purification methods, or it can be a recombinant version of OmpC that is produced in heterologous cells or in vitro. The coding sequence for S. typhi OmpC is also known in the art (see GenBank Accession No. AL627274.1, in which the complement of nucleotides 21394-22530 represents the coding sequence for OmpC from S. enterica subsp. enterica serovar Typhi strain CT18; and GenBank Accession No. AE014613.1, in which nucleotides 681183-682319 represent the coding sequence for OmpC from S. enterica subsp. enterica serovar Typhi Ty2). A representative example of an OmpC coding sequence is provided in FIG. 1B (SEQ ID NO:22).

The OmpC porin incorporated into the product can be the full-length protein or it can be a substantially full-length protein (for example, a protein comprising a N-terminal and/or C-terminal deletion of about 25 amino acids or less, about 20 amino acids or less, about 15 amino acids or less, or about 10 amino acids or less) that retains the adjuvant activity of the wild-type porin. The full-length protein can be the precursor form of OmpC (for example, as shown in FIG. 1A [SEQ ID NO:1]) or the mature (processed) form of OmpC in which the N-terminal leader (or signal) sequence has been removed (for example, the sequence represented by amino acids 22-378 of SEQ ID NO:1).

One skilled in the art will appreciate that the sequence of the OmpC porin incorporated in the product may be varied slightly from the wild-type sequence (i.e. it may be a modified or “variant sequence”) without affecting the ability of the protein to function as an adjuvant. For example, the OmpC porin may comprise one or more mutations, such as, amino acid insertions, deletions or substitutions, provided that the porin retains its ability to act as an adjuvant. As is known in the art, native OmpC is a “beta-barrel” structure with long external loops and shorter internal (periplasmic) turns. In accordance with one embodiment of the invention in which the OmpC comprises a variant sequence, the OmpC variant retains a beta-barrel conformation.

When the OmpC comprises a variant sequence that contains an insertion or deletion, the insertion or deletion in general comprises 20 amino acids or less. In one embodiment of the invention, when the OmpC comprises a variant sequence that contains an insertion or deletion, the insertion or deletion comprises 15 amino acids or less. In another embodiment, when the OmpC comprises a variant sequence that contains an insertion or deletion, the insertion or deletion comprises 10 amino acids or less.

When the OmpC comprises a variant sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

As is known in the art, insertions and deletions in the external loop regions of OmpC are well-tolerated (see, for example, Vega et al., Immunology (2003) 110:206-16). In one embodiment of the invention, the amino acid sequence of the OmpC porin incorporated in the product is a variant sequence comprising an insertion, deletion or substitution in an external loop. In one embodiment of the invention, the amino acid sequence of the OmpC porin incorporated in the product is a variant sequence comprising an insertion or deletion in an external loop in which the insertion or deletion comprises 20 amino acids or less. In another embodiment of the invention, the amino acid sequence of the OmpC porin incorporated in the product is a variant sequence comprising one or more conservative substitutions. In another embodiment of the invention, the amino acid sequence of the OmpC porin incorporated in the product is a variant sequence comprising one or more conservative substitutions in a beta-strand region.

As shown in Table 1, while the sequences of the S. typhi OmpC porin and OmpC orthologues from other enterobacteria are fairly highly conserved, the sequences Salmonella spp. are very highly conserved. In general, the amino acid sequences of OmpC porins from other species of Salmonella show at least 95% sequence identity with OmpC from S. typhi. Accordingly, one embodiment of the invention provides for the inclusion of an OmpC porin from a Salmonella species other than S. typhi as the OmpC component of the product. Additional examples to those provided in Table 1 include, but are not limited to, OmpC from S. enterica serovar Typhimurium (GenBank Accession No. 16761195); OmpC from S. enterica serovar Typhi (GenBank Accession No. 47797); OmpC from S. enterica serovar Minnesota (GenBank Accession No. 8953564); OmpC from S. enterica serovar Dublin (GenBank Accession No. 19743624) and OmpC from S. enterica serovar Gallinarum (GenBank Accession No. 19743622). In those embodiments of the invention in which the product is for use as a multivalent vaccine that provides protection against S. typhi and one or more other disease-causing agents, the OmpC included in the product is S. typhi OmpC, or a modified version thereof.

TABLE 1
Sequence Identity of OmpC and OmpC Orthologues
from Various Enterobacteria

| | | | % |
|---|---|---|---|
| Organism | Protein | Reference | Identity¹ |
| Salmonella typhimurium | OmpC | POA263 | 100 |
| LT2 | | | |
| Salmonella bongori | ORF_2828 | coliBase²: | 99 |
| | (Putative OmpC) | GL026809³ | |
| Salmonella enteritidis | ORF_1402 | coliBase²: | 98 |
| PT4 | (Putative OmpC) | GL063386³ | |
| Salmonella gallinarum | ORF_222 | coliBase²: | 98 |
| 287/91 | (Putative OmpC) | GL064166³ | |
| Escherichia coli O157: | OmpC | Q8XE41 | 80 |
| H7 EDL933 | | | |
| Shigella dysenteriae | ORF_14 | coliBase²: | 78 |
| M131649 (M131)] | (Putative OmpC) | GL018139³ | |
| Shigella flexneri | Omp1b | Q83QU7 | 78 |
| 2a 2457T | | | |

¹% identity is relative to the S. typhi OmpC protein (GenBank Accession No. P0A264) and was determined using the BLASTP 2.2.3 [Apr. 24, 2002] program (Altschul, S. F., et al, (1997), Nucleic Acids Res. 25: 3389-3402).
²Nucleic Acids Research, 2004, Vol. 32, Database issue D296-D299.
³GL numbers as of Jan. 23, 2007.

In another embodiment of the invention, the OmpC porin included in the product is a full-length or substantially full-length OmpC that has an amino acid sequence that has 95% or greater sequence identity with the sequence of the S. typhi OmpC porin as shown in FIG. 1A [SEQ ID NO:1]. In another embodiment, the OmpC porin included in the product is a full-length or substantially full-length OmpC that has an amino acid sequence that has 96% or greater sequence identity with the sequence of the S. typhi OmpC porin as shown in FIG. 1A [SEQ ID NO:1]. In a further embodiment of the invention, the OmpC porin included in the product is a full-length or substantially full-length OmpC that has an amino acid sequence that has 97% or greater sequence identity with the sequence of the S. typhi OmpC porin as shown in FIG. 1A [SEQ ID NO:1]. In other embodiments, the OmpC porin included in the product is a full-length or substantially full-length OmpC that has an amino acid sequence that has 98% or greater sequence identity, or 99% or greater sequence identity with the sequence of the S. typhi OmpC porin as shown in FIG. 1A [SEQ ID NO:1].

### OmpF Porin

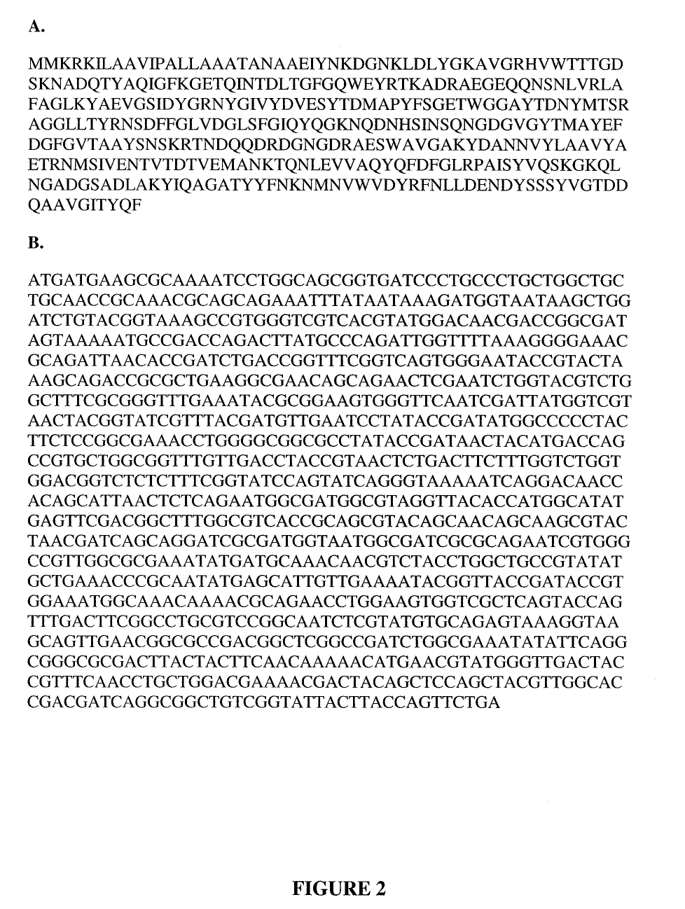

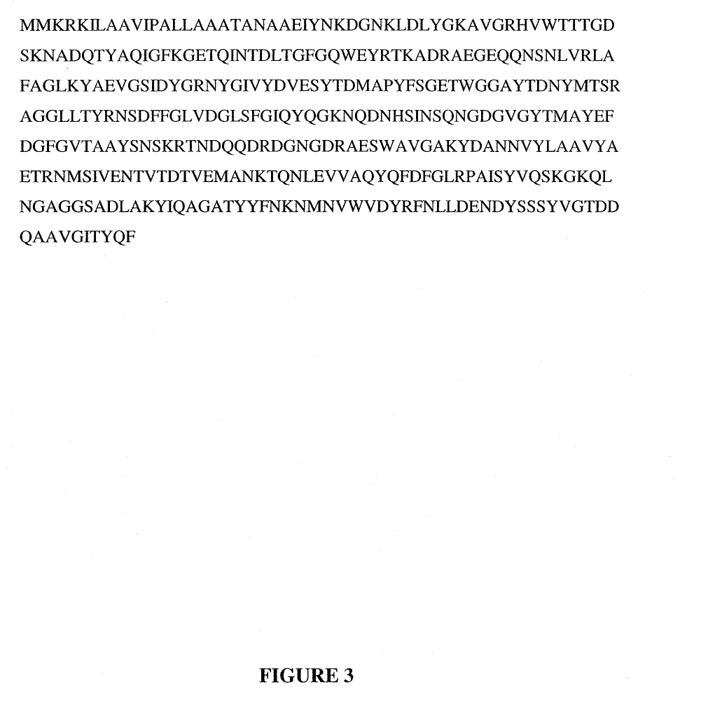

The sequences of OmpF porins from various S. typhi strains are known in the art and are readily accessible, for example, from GenBank database maintained by the NCBI. For example, GenBank Accession No. CAD05399 (SEQ ID NO:2; also shown in FIG. 2A) and GenBank Accession No. NP_{—}455485.1: OmpF precursor protein (S. enterica subsp. enterica serovar Typhi CT18); GenBank Accession No. AA069550.1, GenBank Accession No. NP_{—}805701.1 and GenBank Accession No. Q56113.2: OmpF precursor protein (S. enterica subsp. enterica serovar Typhi Ty2); GenBank Accession No. CAA61905.1 (SEQ ID NO:21; also shown in FIG. 3): OmpF protein (S. typhi); and GenBank Accession No. AAG09474: outer membrane protein F precursor (S. typhi).

The OmpF porin for use in the products according to the invention can be obtained from Salmonella typhi by standard purification methods, or it can be a recombinant version of OmpF that is produced in heterologous cells or in vitro. The coding sequence for S. typhi OmpF is also known in the art (see GenBank Accession No. AL627268.1, in which the complement of nucleotides 241298-242389 represents the coding sequence for OmpF from S. enterica subsp. enterica serovar Typhi strain CT18; and GenBank Accession No. AE014613.1, in which nucleotides 1979688-1980779 represent the coding sequence for OmpF from S. enterica subsp. enterica serovar Typhi Ty2). A representative example of an OmpC coding sequence is provided in FIG. 2B (SEQ ID NO:23).

The OmpF porin incorporated into the product can be the full-length protein or it can be a substantially full-length protein (for example, a protein comprising a N-terminal and/or C-terminal deletion of about 25 amino acids or less, about 20 amino acids or less, about 15 amino acids or less, or about 10 amino acids or less) that retains the adjuvant activity of the wild-type porin. The full-length protein can be the precursor form of OmpF (for example, as shown in FIG. 2A or 3 [SEQ ID NO: 2 or 21]) or the mature (processed) form of OmpF in which the leader (or signal sequence has been removed (for example, the sequence represented by amino acids 23-363 of SEQ ID NO:2).

One skilled in the art will appreciate that the sequence of the OmpF porin incorporated in the product may be varied slightly from the wild-type sequence (i.e. it may be a modified or “variant sequence”) without affecting the ability of the protein to function as an adjuvant. For example, the OmpF porin may comprise one or more mutations, such as, amino acid insertions, deletions or substitutions, provided that the porin retains its ability to act as an adjuvant. As is known in the art, native OmpF is a “beta-barrel” structure with long external loops and shorter internal (periplasmic) turns. In one embodiment, when the OmpF comprises a variant sequence, it also retains a beta-barrel conformation.

When the OmpF comprises a variant sequence that contains an insertion or deletion, the insertion or deletion in general comprises 20 amino acids or less. In one embodiment of the invention, when the OmpF comprises a variant sequence that contains an insertion or deletion, the insertion or deletion comprises 15 amino acids or less. In another embodiment, when the OmpF comprises a variant sequence that contains an insertion or deletion, the insertion or deletion comprises 10 amino acids or less.

When the OmpF comprises a variant sequence that contains one or more amino acid substitutions, these can be “conservative” substitutions or “non-conservative” substitutions, as described above for OmpC. In one embodiment of the invention, the amino acid sequence of the OmpF porin incorporated in the product is a variant sequence comprising an insertion, deletion or substitution in an external loop. In one embodiment of the invention, the amino acid sequence of the OmpF porin incorporated in the product is a variant sequence comprising an insertion or deletion in an external loop in which the insertion or deletion comprises 20 amino acids or less. In another embodiment of the invention, the amino acid sequence of the OmpF porin incorporated in the product is a variant sequence comprising one or more conservative substitutions. In another embodiment of the invention, the amino acid sequence of the OmpF porin incorporated in the product is a variant sequence comprising one or more conservative substitutions in a beta-strand region.

As shown in Table 2, while the sequences of the S. typhi OmpF porin and OmpF orthologues from other enterobacteria are fairly highly conserved, the sequences Salmonella spp. are very highly conserved. In general, the amino acid sequences of OmpF porins from other species of Salmonella show at least 95% sequence identity with OmpF from S. typhi. Accordingly, one embodiment of the invention provides for the inclusion of an OmpF porin from a Salmonella species other than S. typhi as the OmpF component of the product. In those embodiments of the invention in which the product is for use as a multivalent vaccine that provides protection against S. typhi and one or more other disease-causing agents, the OmpF included in the product is S. typhi OmpF, or a modified version thereof.

TABLE 2
Sequence Identity of OmpF and OmpF Orthologues
from Various Enterobacteria

| | | | % |
|---|---|---|---|
| Organism | Protein | Reference | Identity¹ |
| Salmonella enteritidis | ORF_34 | coliBase²: | 100 |
| PT4 | | GL060731³ | |
| Salmonella gallinarum | ORF_21 | coliBase²: | 99 |
| 287/91 | | GL069216³ | |
| Salmonella typhimurium | ORF_287 | coliBase²: | 99 |
| DT104 | | GL0044362³ | |
| Salmonella bongori | ORF_1160 | coliBase²: | 98 |
| | | GL025398³ | |
| Escherichia coli | ORF_2 | coliBase²: | 58 |
| DH10B | | GL037694³ | |
| Shigella flexneri | OmpF | Q83RY7 | 58 |
| 2a 2457T | | | |

¹% identity is relative to the S. typhi OmpF protein (GenBank Accession No. CAD05399) and was determined using the BLASTP 2.2.3 [Apr. 24, 2002] program (Altschul, S. F., et al., (1997), Nucleic Acids Res. 25: 3389-3402.
²Nucleic Acids Research, 2004, Vol. 32, Database issue D296-D299.
³GL numbers as of Jan. 23, 2007.

In another embodiment of the invention, the OmpF porin included in the product is a full-length or substantially full-length OmpF that has an amino acid sequence that has 95% or greater sequence identity with the sequence of the S. typhi OmpF porin as shown in FIG. 2A [SEQ ID NO:2]. In another embodiment, the OmpF porin included in the product is a full-length or substantially full-length OmpF that has an amino acid sequence that has 96% or greater sequence identity with the sequence of the S. typhi OmpF porin as shown in FIG. 2A [SEQ ID NO:2]. In a further embodiment of the invention, the OmpF porin included in the product is a full-length or substantially full-length OmpF that has an amino acid sequence that has 97% or greater sequence identity with the sequence of the S. typhi OmpF porin as shown in FIG. 2A [SEQ ID NO:2]. In other embodiments, the OmpF porin included in the product is a full-length or substantially full-length OmpF that has an amino acid sequence that has 98% or greater sequence identity, or 99% or greater sequence identity with the sequence of the S. typhi OmpF porin as shown in FIG. 2A [SEQ ID NO:2].

### Preparation of OmpC and OmpF

The OmpC and/or OmpF porins can be purified from S. typhi using standard techniques known in the art. An example of such a technique has been described by Salazar-Gonzalez et al. in Immunol. Lett. (2004) 93:115-122 (herein expressly incorporated by reference in its entirety). A representative method is also provided herein as Example 1. In order to obtain a preparation of OmpC that is substantially free of OmpF, an OmpF knockout mutant strain of S. typhi may be used. For example, Salmonella strain STYF302 (ΔompF Km^{R}) (Martinez-Flores et al., J. Bacteriol. (1999) 181:556-562). Similarly, in order to obtain a preparation of OmpF that is substantially free of OmpC, an OmpC knockout mutant strain of S. typhi may be used. For example, Salmonella strain STYC171 (ΔompC Km^{R}) (Martinez-Flores et al., ibid.).

In general, porin purification from S. typhi involves first growing the bacteria in a suitable medium under suitable conditions until an acceptable density has been achieved, for example, to an OD₅₄₀ of between about 0.8 and about 1.5. The cells are harvested and lysed and the OmpC and/or OmpF porin extracted by a series of centrifugation and homogenisation steps. The porin(s) can be further purified by standard chromatography, for example, fast protein liquid chromatography (FPLC) or medium-pressure liquid chromatography (MPLC), using size-exclusion, gel filtration or other medium. Both OmpC and OmpF preparations are generally stable and can be stored at 4° C. for extended periods of time, for example, for periods of 4 weeks or more. In one embodiment of the invention in which OmpC and OmpF were prepared essentially as described in Example 1, the porin preparation was stable at 4° C. for one year or more.

The porins can also be prepared by standard genetic engineering techniques by the skilled worker provided with the sequence of the wild-type protein(s). Methods of cloning and expressing recombinant proteins are well known in the art (see, for example, Ausubel et al. (1994 & updates) Current Protocols in Molecular Biology, John Wiley & Sons, New York), as are the sequences of the wild-type OmpC and OmpF proteins (see, for example, SEQ ID NOs:1 and 2).

Isolation and cloning of the nucleic acid sequence encoding the OmpC or OmpF wild-type protein can be achieved using standard techniques (see, for example, Ausubel et al., ibid.). For example, the nucleic acid sequence can be obtained directly from S. typhi by standard techniques (for example, by PCR-based techniques). The nucleic acid sequence encoding the relevant porin protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The porin can then be expressed and purified using standard techniques.

When desired, the nucleic acid sequence encoding the OmpC or OmpF porin protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

One of ordinary skill in the art will appreciate that the DNA encoding the porin protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the porin protein. Examples of regulatory elements that can be incorporated into the vector include, but are not limited to, promoters, enhancers, terminators, and polyadenylation signals. The present invention, therefore, provides for vectors comprising a regulatory element operatively linked to a nucleic acid sequence encoding a recombinant OmpC or OmpF protein. One skilled in the art will appreciate that selection of suitable regulatory elements is dependent on the host cell chosen for expression of the porin protein and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids encoded by the heterologous nucleic acid sequence can be removed from the expressed porin protein prior to use according to methods known in the art. Alternatively, the amino acids corresponding to expression of heterologous nucleic acid sequences can be retained on the porin protein provided that they do not interfere with its adjuvant activity.

The expression vector can be introduced into a suitable host cell by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the porin protein will be dependent upon the vector chosen. Examples of host cells include, but are not limited to, bacterial, yeast, insect, plant and mammalian cells. The porin proteins can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; insect cells or plant cells). When the selected host is a bacterial host, the use of a porin-deficient strain of bacteria, for example a porin-deficient E. coli strain such as the E. coli UH302 strain, can facilitate the subsequent purification of the OmpC or OmpF porin.

The recombinant porin protein can be isolated from the host cells by standard methods such as those described above for the wild-type proteins or following other published protocols (see, for example, Arockiasamy, et al. Anal. Biochem. (2000) 283:64-70; Vega, et al. Immunology (2003) 110:206-216). The protein can be further purified by standard techniques, such as chromatography, to remove contaminating host cell proteins or other compounds, such as LPS. In one embodiment of the present invention, the porin protein is purified to remove LPS.

### Antigenic Material

As noted above, when the product of the invention is a combination product, it comprises antigenic material. The immunogenic compositions of the invention can be used to potentiate the effects of separately formulated antigenic material or may themselves comprise antigenic material. The antigenic material can be purified or partially purified. The antigenic material can be in the form of one or more purified or partially purified antigens, for example, antigenic proteins or protein fragments, or whole cells or fragments of whole cells. Alternatively, the antigenic material can be provided in the form of a known vaccine, for example, a commercially available vaccine.

A wide variety of antigenic material suitable for the development of vaccines is known in the art. Appropriate antigenic material for inclusion in the products of the invention can be readily selected by one skilled in the art based on, for example, the desired end use of the product such as the disease or disorder against which it is to be directed, the format of composition, whether the composition is intended for use as a multivalent or monovalent vaccine and/or the animal to which it is to be administered.

For example, the antigenic material can be derived from an agent capable of causing a disease or disorder in an animal, such as a cancer, infectious disease, allergic reaction, or autoimmune disease, or it can be antigenic material suitable for use to induce an immune response against drugs, hormones or a toxin-associated disease or disorder. The antigenic material may be derived from a pathogen known in the art, such as, for example, a bacterium, virus, protozoan, fungus, parasite, or infectious particle, such as a prion, or it may be a tumour-associated antigen, a self-antigen or an allergen.

By way of example, antigenic material may be derived from known causative agents responsible for diseases such as diptheria (e.g. Corynebacterium diphtheriae), pertussis (e.g. Bordetella pertussis), tetanus (e.g. Clostridium tetani), tuberculosis (e.g. Mycobacterium tuberculosis), bacterial or fungal pneumonia, cholera (e.g. Vibrio cholerae), typhoid fever (e.g. S. typhi), plague, shigellosis (e.g. Shigella dysenteriae serotype 1 (S. dysenteriae 1)), Salmonellosis, Legionnaire's disease (e.g. Legionella pneumophila), Lyme disease, leprosy (e.g. Mycobacterium leprae), malaria (e.g. Plasmodium falciparium), Hookworm, Onchocerciasis, Schistosomiasis, Trypamasomialsis, leishmaniasis, giardia (e.g. Giardia lamblia), Amoebiasis (e.g. Entamoeba histolytica), Filariasis, Borrelia, Trichinosis, influenza, hepatitis B and C, meningococcal meningitis, community acquired pneumonia, chickenpox, rubella, mumps, measles, AIDS, dengue respiratory infections, diarrhoeal diseases, tropical parasitic diseases, sexually transmitted diseases and chlamydia infections. Antigenic material may also be derived from causative agents responsible for new emerging, re-emerging diseases or bioterrorism diseases such as: SARS infection, Vancomycin-resistant S. aureus infections, West Nile Virus infections, Cryptosporidiosis, Hanta virus infections, Epstein Barr virus infections, Cytomegalovirus infections, H5N1 influenza, Enterovirus 71 infections, E. coli. O157:H7 infections, human monkey pox, Lyme disease, Cyclosporiasis, Hendra virus infections, Nipah virus infections, Rift Valley fever, Marburg haemorrhagic fever, Whitewater arrollo virus infections and Anthrax.

The size of the antigenic material for incorporation into the immunogenic compositions is not critical to the invention and the selected antigenic material can thus vary in size. The antigenic material may be, for example, a peptide, a protein, a nucleic acid, a polysaccharide, a lipid, a small molecule, or a combination thereof up to and including a whole pathogen or a portion thereof, for example, a live, inactivated or attenuated version of a pathogen.

When the antigenic material for incorporation into the product of the invention comprises more than one antigen, the antigens selected for inclusion in the product can be derived from a single source, such that the product is a monovalent product, or can be derived from a plurality of sources, such that the product is a multivalent product. The antigens can each have a single epitope capable of triggering a specific immune response, or each antigen may comprise more than one epitope.

The antigenic material may comprise epitopes recognised by surface structures on T cells, B cells, NK cells, dendritic cells, macrophages, polymorphonuclear leukocytes, Class I or Class II APC associated cell surface structures, or a combination thereof.

Antigenic material for inclusion in the products of the invention may also be selected from pathogens or other sources of interest by art known methods and screened for their ability to induce an immune response in an animal using standard immunological techniques known in the art. For example, methods for prediction of epitopes within an antigenic protein are described in Nussinov R and Wolfson H J, Comb Chem High Throughput Screen (1999) 2(5):261, and methods of predicting CTL epitopes are described in Rothbard et al., EMBO J. (1988) 7:93-100 and in de Groot M S et al., Vaccine (2001) 19(31):4385-95. Other methods are described in Rammensee H-G. et al., Immunogenetics (1995) 41:178-228 and Schirle M et al., Eur J Immunol (2000) 30(18):2216-2225.

Useful viral antigenic material for example, includes antigenic material derived from members of the families Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxyiridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). In one embodiment, the products comprise one or more antigens derived from a major viral pathogen such as the various hepatitis viruses, polio virus, human immunodeficiency virus (HIV), various influenza viruses, West Nile virus, respiratory syncytial virus, rabies virus, human papilloma virus (HPV), Epstein Barr virus (EBV), polyoma virus, or SARS coronavirus.

Viral antigenic material derived from the hepatitis viruses, including hepatitis A virus (HAV), hepatitis B virus (HBV), hepatitis C virus (HCV), delta hepatitis virus (HDV), hepatitis E virus (HEV) and hepatitis G virus (HGV), are known in the art. For example, antigens can be derived from HCV core protein, E1 protein, E2 protein, NS3 protein, NS4 protein or NS5 protein; from HBV HbsAg antigen or HBV core antigen, and from HDV delta-antigen (see, for example, U.S. Pat. No. 5,378,814). U.S. Pat. Nos. 6,596,476; 6,592,871; 6,183,949; 6,235,284; 6,780,967; 5,981,286; 5,910,404; 6,613,530; 6,709,828; 6,667,387; 6,007,982; 6,165,730; 6,649,735 and 6,576,417, for example, describe various antigens based on HCV core protein.

Non-limiting examples of known antigens from the herpesvirus family include those derived from herpes simplex virus (HSV) types 1 and 2, such as HSV-1 and HSV-2 glycoproteins gB, gD and gH.

Non-limiting examples of HIV antigens include antigens derived from gp120, antigens derived from various envelope proteins such as gp160 and gp41, gag antigens such as p24gag and p55gag, as well as proteins derived from the pol, env, tat, vif rev, nef vpr, vpu and LTR regions of HIV. The sequences of gp120 from a multitude of HIV-1 and HIV-2 isolates, including members of the various genetic subtypes of HIV are known (see, for example, Myers et al., Los Alamos Database, Los Alamos National Laboratory, Los Alamos, N. Mex. (1992); and Modrow et al., J. Virol. (1987) 61:570-578).

Non-limiting examples of other viral antigenic material includes antigenic material from varicella zoster virus (VZV), Epstein-Barr virus (EBV) and cytomegalovirus (CMV) including CMV gB and gH; and antigens from other human herpesviruses such as HHV6 and HHV7 (see, for example Chee et al. (1990) Cytomegaloviruses (J. K. McDougall, ed., Springer-Verlag, pp. 125-169; McGeoch et al. (1988) J. Gen. Virol. 69:1531-1574; U.S. Pat. No. 5,171,568; Baer et al. (1984) Nature 310:207-211; and Davison et al. (1986) J. Gen. Virol. 67:1759-1816.)

Antigenic material can also be derived from the influenza virus, for example, the antigenic material can be attenuated, killed or inactivated influenza virus. Alternatively, the antigenic material from the influenza virus can be derived from the haemagglutinin (HA), neuramidase (NA), nucleoprotein (NP), M1 or M2 proteins. The sequences of these proteins are known in the art and are readily accessible from GenBank database maintained by the National Center for Biotechnology Information (NCBI). Suitable antigenic fragments of HA, NP and the matrix proteins include, but are not limited to, the haemagglutinin epitopes: HA 91-108, HA 307-319 and HA 306-324 (Rothbard, Cell, 1988, 52:515-523), HA 458-467 (J. Immunol. 1997, 159(10): 4753-61), HA 213-227, HA 241-255, HA 529-543 and HA 533-547 (Gao, W. et al., J. Virol., 2006, 80:1959-1964); the nucleoprotein epitopes: NP 206-229 (Brett, 1991, J. Immunol. 147:984-991), NP335-350 and NP380-393 (Dyer and Middleton, 1993, In: Histocompatibility testing, a practical approach (Ed.: Rickwood, D. and Hames, B. D.) IRL Press, Oxford, p. 292; Gulukota and DeLisi, 1996, Genetic Analysis: Biomolecular Engineering, 13:81), NP 305-313 (DiBrino, 1993, PNAS 90:1508-12); NP 384-394 (Kvist, 1991, Nature 348:446-448); NP 89-101 (Cerundolo, 1991, Proc. R. Soc. Lon. 244:169-7); NP 91-99 (Silver et al, 1993, Nature 360: 367-369); NP 380-388 (Suhrbier, 1993, J. Immunology 79:171-173); NP 44-52 and NP 265-273 (DiBrino, 1993, ibid.); and NP 365-380 (Townsend, 1986, Cell 44:959-968); the matrix protein (M1) epitopes: M1 2-22, M1 2-12, M1 3-11, M1 3-12, M1 41-51, M1 50-59, M1 51-59, M1 134-142, M1 145-155, M1 164-172, M1 164-173 (all described by Nijman, 1993, Eur. J. Immunol. 23:1215-1219); M1 17-31, M1 55-73, M1 57-68 (Carreno, 1992, Mol Immunol 29:1131-1140); M1 27-35, M1 232-240 (DiBrino, 1993, ibid.), M1 59-68 and M1 60-68 (Eur. J. Immunol. 1994, 24(3): 777-80); and M1 128-135 (Eur. J. Immunol. 1996, 26(2): 335-39).

Other related antigenic regions and epitopes of the influenza virus are also known. For example, fragments of the influenza ion channel protein (M2), including the M2e peptide (the extracellular domain of M2). The sequence of this peptide is highly conserved across different strains of influenza. An example of a M2e peptide sequence is shown in Table 3 as SEQ ID NO:3. Variants of this sequence have been identified and non-limiting examples are also shown in Table 3.

TABLE 3
M2e Peptide and Variations Thereof

| Region | | | SEQ ID |
|---|---|---|---|
| of M2 | Sequence | Viral Strain | NO |
| 2-24 | SLLTEVETPIRNEWGCRCNDSSD | Human H1N1 e.g. | 3 |
| | | A/USRR/90/77 and | |
| | | A/WSN/33 | |
| 2-24 | SLLTEVETPIRNEWGCRCNGSSD | N/A* | 4 |
| 2-24 | SLLTEVETPTKNEWDCRCNDSSD | N/A* | 5 |
| 2-24 | SLLTEVETPTRNGWECKCSDSSD | Equine H3N8 | 6 |
| | | A/equine/Massachussetts/ | |
| | | 213/2003 | |
| 2-24 | SLLTEVETPTRNEWECRCSDSSD | H5N1 A/Vietnam/1196/04 | 7 |
| 1-24 | MSLLTEVETPIRNEWGCRCNDSSD | Human H1N1 e.g. | 8 |
| | | A/USRR/90/77 and | |
| | | A/WSN/33 | |
| 1-24 | HSLLTEVETPTRNEWECRCSDSSD | Avian H5N1 | 9 |
| | | A/Vietnam/1196/04 | |
| 1-24 | MSLLTEVETPTRNGWECKCSDSSD | H3N8, Horse-Dog | 10 |
| | | A/equine/Massachussetts/ | |
| | | 213/2003 | |
| 1-24 | MSLLTEVETPTRNGWGCRCSDSSD | H9N2, | 11 |
| | | A/chicken/Osaka/aq69/2001 | |
| 1-24 | MSLLTEVETPTRNEWGCRCSDSSD | Mutant H1N1 I/T | 12 |

*see U.S. patent application No. 2006/0246092

The entire M2e sequence or a partial M2e sequence may be used, for example, a partial sequence that is conserved across the variants, such as fragments within the region defined by amino acids 2 to 10, or the conserved epitope EVETPIRN [SEQ ID NO:13] (amino acids 6-13 of the M2e sequence). The 6-13 epitope has been found to be invariable in 84% of human influenza A strains available in GenBank. Variants of this sequence that were also identified include EVETLTRN [SEQ ID NO:14] (9.6%), EVETPIRS [SEQ ID NO:15] (2.3%), EVETPTRN [SEQ ID NO:16] (1.1%), EVETPTKN [SEQ ID NO:17] (1.1%) and EVDTLTRN [SEQ ID NO:18], EVETPIRK [SEQ ID NO:19] and EVETLTKN [SEQ ID NO:20] (0.6% each) (see Zou, P., et al., 2005, Int Immunopharmacology, 5:631-635; Liu et al. 2005, Microbes and Infection, 7:171-177).

As is known in the art, there are three genera of influenza virus: types A, B and C. Antigenic material for incorporation into or use with the products of the invention may be derived from influenza virus type A, type B or type C, or a combination thereof. In one embodiment, the antigenic material for incorporation into or use with the products of the invention is derived from influenza virus type A or type B, or a combination thereof. In addition, many strains of influenza are presently in existence. Important examples include, but are not limited to, those listed in Table 3. Antigenic material for incorporation into or use with the products of the invention may be derived from one strain of influenza virus or multiple strains, for example, between two and five strains, in order to provide a broader spectrum of protection. In one embodiment, antigenic material for incorporation into or use with the products of the invention is derived from multiple strains of influenza virus.

Other useful antigenic material includes live, attenuated and inactivated viruses such as inactivated polio virus (Jiang et al., J. Biol. Stand., (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNE™ acellular DTP, Wyeth-Lederle Vaccines and Pediatrics).

Antigenic material can also be derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease, as are known in the art.

Useful bacterial antigenic material includes, for example, whole inactivated cells, superficial bacterial antigenic components, such as lipopolysaccharides, capsular antigens (proteinacious or polysaccharide in nature), or flagellar components.

Examples of antigenic material derived from gram-negative bacteria of the family Enterobacteriaceae includes, but is not limited to, the S. typhi Vi (capsular polysaccharide) antigen, the E. coli K and CFA (capsular component) antigens and the E. coli fimbrial adhesin antigens (K88 and K99). Examples of antigenic proteins include the outer membrane proteins related to OmpC and OmpF porins such as the S. typhi iron-regulated outer membrane protein (IROMP, Sood et al., 2005, Mol Cell Biochem 273:69-78), and heat shock proteins (HSPS) including, but not limited to S. typhi HSP40 (Sagi et al., 2006, Vaccine 24:7135-7141). Non-limiting examples of antigenic porins include non-Salmonella OmpC and OmpF, which are found in numerous Escherichia species. Orthologues of OmpC and OmpF are also found in other Enterobacteriaceae and are suitable antigenic proteins for the purposes of the present invention. In addition, Omp1B (Shigella flexneri), OmpC2 (Yersinia pestis), OmpD (S. enterica), OmpK36 (Klebsiella pneumoniae), OmpN (E. coli) and OmpS (S. enterica) may be suitable, based on conserved regions of sequences found in the porin proteins of the Enterobacteriaceae family (Diaz-Quinonez et al., 2004, Infect. and Immunity 72:3059-3062).

The sequences of antigenic proteins from various enterobacteria are known in the art and are readily accessible from GenBank database maintained by the National Center for Biotechnology Information (NCBI). For example, GenBank Accession No. 26248604: OmpC (E. coli); GenBank Accession No. 24113600: Omp1B (Shigella flexneri); GenBank Accession No. 16764875: OmpC2 (Yersinia pestis); GenBank Accession No. 16764916: OmpD (S. enterica Serovar Typhimurium); GenBank Accession No. 151149831: OmpK36 (Klebsiella pneumonie); GenBank Accession No. 3273514: OmpN (E. coli), and GenBank Accession No. 16760442: OmpS (S. enterica serovar Typhi).

Various tumour-associated antigens are known in the art. Representative examples include, but are not limited to, Her2 (breast cancer); GD2 (neuroblastoma); EGF-R (malignant glioblastoma); CEA (medullary thyroid cancer); CD52 (leukemia); human melanoma protein gp100; human melanoma protein melan-A/MART-1; NA17-A nt protein; p53 protein; various MAGEs (melanoma associated antigen E), including MAGE 1, MAGE 2, MAGE 3 (HLA-A1 peptide) and MAGE 4; various tyrosinases (HLA-A2 peptide); mutant ras; p97 melanoma antigen; Ras peptide and p53 peptide associated with advanced cancers; the HPV 16/18 and E6/E7 antigens associated with cervical cancers; MUC1-KLH antigen associated with breast carcinoma; CEA (carcinoembryonic antigen) associated with colorectal cancer, and the PSA antigen associated with prostate cancer.

Allergens that can be used as antigenic material include, but are not limited to, allergens from pollens, animal dander, grasses, moulds, dusts, antibiotics, stinging insect venoms, as well as a variety of environmental, drug and food allergens. Common tree allergens include pollens from cottonwood, popular, ash, birch, maple, oak, elm, hickory, and pecan trees. Common plant allergens include those from rye, ragweed, English plantain, sorrel-dock and pigweed, and plant contact allergens include those from poison oak, poison ivy and nettles. Common grass allergens include Timothy, Johnson, Bermuda, fescue and bluegrass allergens. Common allergens can also be obtained from moulds or fungi such as Alternaria, Fusarium, Hormodendrum, Aspergillus, Micropolyspora, Mucor and thermophilic actinomycetes. Penicillin, sulfonamides and tetracycline are common antibiotic allergens. Epidermal allergens can be obtained from house or organic dusts (typically fungal in origin), from insects such as house mites (Dermalphagoides pterosinyssis), or from animal sources such as feathers, and cat and dog dander. Common food allergens include milk and cheese (diary), egg, wheat, nut (for example, peanut), seafood (for example, shellfish), pea, bean and gluten allergens. Common drug allergens include local anesthetic and salicylate allergens, and common insect allergens include bee, hornet, wasp and ant venom, and cockroach calyx allergens.

Particularly well characterized allergens include, but are not limited to, the dust mite allergens Der pI and Der pII (see, Chua, et al., J. Exp. Med., 167:175 182, 1988; and, Chua, et al., Int. Arch. Allergy Appl. Immunol., (1990) 91:124-129), T cell epitope peptides of the Der pII allergen (see, Joost van Neerven, et al., J. Immunol., (1993) 151:2326-2335), the highly abundant Antigen E (Amb aI) ragweed pollen allergen (see, Rafnar, et al., J. Biol. Chem., (1991) 266:1229-1236), phospholipase A2 (bee venom) allergen and T cell epitopes therein (see, Dhillon, et al., J. Allergy Clin. Immunol., (1992) 42), white birch pollen (Betvl) (see, Breiteneder, et al., EMBO, (1989) 8:1935-1938), the Fel dI major domestic cat allergen (see, Rogers, et al., Mol. Immunol., (1993) 30:559-568), tree pollen (see, Elsayed et al., Scand. J. Clin. Lab. Invest. Suppl., (1991) 204:17-31) and the multi-epitopic recombinant grass allergen rKBG8.3 (Cao et al. Immunology (1997) 90:46-51). These and other suitable allergens are commercially available and/or can be readily prepared following known techniques.

Antigenic material relating to conditions associated with self antigens is also known to those of ordinary skill in the art. Representative examples of such antigenic material includes, but are not limited to, lymphotoxins, lymphotoxin receptors, receptor activator of nuclear factor kB ligand (RANKL), vascular endothelial growth factor (VEGF), vascular endothelial growth factor receptor (VEGF-R), interleukin-5, interleukin-17, interleukin-13, CCL21, CXCL12, SDF-1, MCP-1, endoglin, resistin, GHRH, LHRH, TRH, MIF, eotaxin, bradykinin, BLC, Tumour Necrosis Factor alpha and amyloid beta peptide, as well as fragments of each which can be used to elicit immunological responses.

Toxins that can be used as antigenic material are generally the natural products of toxic plants, animals, and microorganisms, or fragments of these compounds. Such compounds include, for example, aflatoxin, ciguautera toxin, pertussis toxin and tetrodotoxin.

Antigenic material useful in relation to recreational drug addiction is known in the art and includes, for example, opioids and morphine derivatives such as codeine, fentanyl, heroin, morphine and opium; stimulants such as amphetamine, cocaine, MDMA (methylenedioxymethamphetamine), methamphetamine, methylphenidate, and nicotine; hallucinogens such as LSD, mescaline and psilocybin; cannabinoids such as hashish and marijuana, other addictive drugs or compounds, and derivatives, by-products, variants and complexes of such compounds.

As noted above, in various embodiments, the antigenic material included in or for use with the product of the invention is a known vaccine composition. Various human vaccines are known in the art and include, but are not limited to, vaccines against:
Bacillus anthracis (anthrax), such as BioThrax® (BioPort Corporation);
Haemophilus influenzae type b (Hib), such as, ActHIB® (Sanofi-aventis), PedvaxHlB® (Merck) and HibTITER® (Wyeth);
hepatitis A, such as, Havrix® (GlaxoSmithKline) and Vaqta® (Merck);
hepatitis B, such as, Engerix-B® (GlaxoSmithKline) and Recombivax HB® (Merck);
Herpes zoster (shingles), such as, Zostavax® (Merck);
human papillomavirus (HPV), such as, Gardasil® (Merck);
influenza, such as, Fluarix® and Fluviral® (GlaxoSmithKline), FluLaval®(ID Biomedical Corp of Quebec); FluMist® (intranasal) (Medimmune), Fluvirin® (Chiron); Fluzone® (Sanofi-aventis) and Influvac™ (Solvay);
Japanese encephalitis, such as, JE-Vax® (Sanofi-aventis);
measles, such as, Attenuvax® (Merck);
Meningococcal meninigitis, such as, Menomune® Meningococcal Polysaccharide (Sanofi-aventis);
mumps, such as, Mumpsvax® (Merck);
pneumococcal disease, such as, Pneumovax 23® Pneumococcal Polysaccharide (Sanofi-aventis) and Prevnar® Pneumococcal Conjugate (Wyeth);
polio, such as, Ipol® (Sanofi-aventis) and Poliovax® (Sanofi-Pasteur);
rabies, such as, BioRab® (BioPort Corporation), RabAvert® (Chiron) and Imovax® Rabies (Sanofi-aventis);
rotavirus, such as, RotaTeq® (Merck);
rubella, such as, Meruvax II® (Merck);
S. typhi (typhoid fever), such as, Typhim Vi® (Sanofi-aventis) and Vivotif®Berna (oral) (Berna);
tuberculosis (BCG), such as, TheraCys® and ImmuCyst® (Sanofi-aventis); TICE® BCG and Oncotice™ (Organon Teknika Corporation); Pacis™; and Mycobax® (Sanofi-Pasteur);
vaccinia (smallpox), such as, Dryvax® (Wyeth);
varicella (chickenpox), such as, Varivax® (Merck);
yellow fever, such as, YF-Vax® (Sanofi-aventis);
hepatitis A/hepatitis B, such as, Twinrix® (GlaxoSmithKline);
hepatitis B and Hib, such as, Comvax® (Merck);
tetanus/Hib, such as, ActHIB® (Sanofi-Pasteur);
diphtheria/Hib, such as, HibTITER® (Wyeth Pharmaceuticals);
Hib/meningitis, such as, PedVaxHIB (Merck & Co);
meningitis/diptheria, such as, Menactra® Meningococcal Conjugate (Sanofi-Pasteur);
tetanus/dipheria (Td), such as, Decavac® (Sanofi-aventis);
diphtheria/tetanus/pertussis (DTaP/DT or DTaP), such as, Daptacel® and Tripedia® (Sanofi-aventis) and Infanrix® (GlaxoSmithKline);
tetanus/diphtheria/pertussis (Tdap), such as, Boostrix® (GlaxoSmithKline) and Adacel® (Sanofi-Pasteur);
DTaP/Hib, such as, TriHIBit® (Sanofi-aventis);
DTaP/polio/hepatitis B, such as Pediarix® (GlaxoSmithKline);
measles/mumps/rubella (MMR), such as, M-M-R II (Merck) and
measles/mumps/rubella/chickenpox, such as, ProQuad® (Merck).

Examples of vaccines for veterinarian use include, but are not limited to, vaccines against Lawsonia intracellularis (for example, Enterisol and Ileitis), Porphyromonas gulae, and P. denticanis (for example, Periovac), Streptococcus equi (for example, Equilis StrepE), Chlamydophila abortus (for example, Ovilis and Enzovax), Mycoplasma synoviae (for example, Vaxsafe MS), Mycoplasma gallisepticum (for example, Vaxsafe MG), Bordetella avium (for example, Art Vax), Actinobacillus pleuropneumoniae (for example, PleuroStar APP), Actinobacillus pleuropneumoniae (for example, Porcilis APP), Salmonella (for example, Megan Vac1 and MeganEgg), Brucella abortus (for example, RB-51), Eimeria spp. (for example, Coccivac, Immucox, Paracox, Advent, and Nobilis Cox ATM), Eimeria spp. (for example, Inovocox), E. tenella (for example, Livacox), Toxoplasma gondii (for example, Ovilis and Toxovax), Pseudorabies virus (for example, Suvaxyn Aujeszky), Classical swine fever virus (for example, Porcilis Pesti and Bayovac CSF E2), Equine influenza virus (for example, PROTEQ-FLU and Recombitek), Newcastle disease virus (for example, Vectormune FP-ND), Avian influenza virus (for example, Poulvac FluFend I AI H5N3 RG), Avian influenza virus (for example, Trovac AI H5), Rabies virus (for example, Raboral and Purevax Feline Rabies), Feline leukemia virus (for example, EURIFEL FeLV), Canine parvovirus 1 (for example, RECOMBITEK Canine Parvo), Canine coronavirus (for example, RECOMBITEK Corona MLV), Canine distemper virus (for example, RECOMBITEK rDistemper and PUREVAXFerret Distemper), IHN virus (for example, Apex-IHN). Other examples of veterinarian vaccines include reproduction control vaccines such as LHRH (for example, Vaxstrate, Improvac, Equito, Canine gonadotropin releasing factor immunotherapeutic, and GonaCon) and Androstenedione (for example, Fecundin, Androvax and Ovastim).

In one embodiment of the invention, the antigenic material included in or for use with the product of the invention is in the form of a pre-formulated influenza vaccine. In general, commercial influenza vaccines comprise inactivated whole virions or split virions. In one embodiment, therefore, the invention provides for products comprising OmpC and/or OmpF and an inactivated whole virion or split virion influenza vaccine. In a specific embodiment, the invention provides for products comprising OmpC and/or OmpF and an inactivated whole split virion influenza vaccine.

Commercially available influenza vaccines are also typically trivalent in that they provide protection against three strains of influenza—in general strains of influenza A and influenza B. For example, for the 2007-2008 season, the strains were A/Solomon Islands/3/2006 (H1N1)-like, A/Wisconsin/67/2005 (H3N2)-like, and B/Malaysia/2506/2004-like; and for the 2008-2009 season the strains were A/Brisbane/59/2007 (H1N1); A/Brisbane/10/2007 (H3N2) and B/Florida/4/2006.

Influenza vaccines that are presently commercially available include, but are not limited to, Fluzone® and Vaxigrip® (Sanofi-aventis), Fluvirin® (Novartis Vaccine), Fluarix®, FluLaval® and Fluviral S/F® (GlaxoSmithKline), Afluria (CSL Biotherapies), FluMist® (MedImmune), and Influvac™ (Solvay Pharma).

### Pharmaceutical Compositions

As noted above, the products of the invention may further optionally comprise a suitable carrier, excipient or the like, and/or other standard components of pharmaceutical compositions that improve the stability, palatability, pharmacokinetics, bioavailability or the like, of the product. In one embodiment of the invention, the pharmaceutical composition comprises OmpC and/or OmpF and is formulated for use as an adjuvant. In another embodiment, the immunogenic composition comprises OmpC and/or OmpF and antigenic material and is formulated for use as a vaccine.

The compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the pharmaceutical composition to the mucous membranes of the nasal passage or nasal cavity of the subject. In one embodiment of the present invention, the compositions are formulated for topical, rectal or parenteral administration or for administration by inhalation or spray, for example by an intranasal route. In another embodiment, the compositions are formulated for parenteral administration. In a further embodiment, compositions are formulated for subcutaneous or intramuscular administration. A non-limiting example of a formulation of OmpC suitable for subcutaneous or intramuscular administration is provided by Salazar-Gonzales, et al., (Immunol. Lett. (2004) 93:115-122).

The compositions preferably comprise an effective amount of OmpC and/or OmpF. The term “effective amount” as used herein refers to an amount of the porin(s) required to produce a detectable immune response in combination with antigenic material. The effective amount for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the present invention, in which the composition comprises OmpC, the unit dose comprises between about 1 μg to about 10 mg of OmpC protein. In another embodiment, in which the composition comprises OmpC, the unit dose comprises between about 1 μg to about 5 mg of OmpC protein. In another embodiment, in which the composition comprises OmpC, the unit dose comprises between about 1 μg to about 2 mg of OmpC protein. In other embodiments, in which the composition comprises OmpC, the unit dose comprises between about 1 μg to about 1 mg, between about 1 μg to about 90 μg, between about 1 μg to about 80 μg, between about 1 μg to about 70 μg, between about 1 μg to about 60 μg or between about 1 μg to about 50 μg of OmpC protein. One or more doses may be used to immunise the animal, and these may be administered on the same day or over the course of several days or weeks.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the immunogenic composition in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the immunogenic composition is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the porin preparation and optional antigenic material in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the porin preparation and optional antigenic material in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the immunogenic composition in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Compositions in accordance with the present invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the porin preparation and optional antigenic material with one or more sweetening agents, for example glycerol, propylene glycol, sorbitol or sucrose. Such formulations can also optionally contain one or more demulcents, preservatives, flavouring agents and/or colouring agents.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the composition in accordance with the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum albumin or skimmed milk) together with a suitable buffer (e.g. phosphate buffer). The pH and exact concentration of the various components of the composition may be adjusted according to well-known parameters.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Testing for Efficacy

The ability of the products of the present invention to potentiate or induce an immune response in an animal can be tested by art-known methods, such as those described below and in the Examples. For example, the product can be administered to a suitable animal model, for example by subcutaneous injection or intranasally, and the development of specific antibodies evaluated by standard techniques, such as Enzyme-Linked Immunosorbent Assay (ELISA).

Cellular immune response can also be assessed by techniques known in the art. For example, the cellular immune response can be determined by evaluating processing and cross-presentation of an epitope comprised by the product to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines (see, for example, Leclerc, D., et al., J. Virol, 2007, 81(3):1319-26).

In order to determine the efficacy of products comprising OmpC and/or OmpF and antigenic material as vaccines, challenge studies can be conducted. Such studies involve the inoculation of groups of a test animal (such as mice) with a product of the invention by standard techniques. Control groups comprising non-inoculated animals and/or animals inoculated with a commercially available vaccine, or other positive control, are set up in parallel. After an appropriate period of time post-vaccination, the animals are challenged with the naturally-occurring substance or organism that contains the antigenic material comprised by the product. Blood samples collected from the animals pre- and post-inoculation, as well as post-challenge are then analyzed for an antibody response to the substance or organism. Suitable tests for the antibody response include, but are not limited to, Western blot analysis and ELISA. The animals can also be monitored for development of the condition associated with the substance or organism. When the product is intended for use as a multivalent product that provides protection against more than one organism, challenge studies that test the ability of the product to protect against each organism should be conducted.

Similarly, products comprising tumour-associated antigens can be tested for their prophylactic effect by inoculation of test animals and subsequent challenge by transplanting cancer cells into the animal, for example subcutaneously, and monitoring tumour development in the animal. Alternatively, the therapeutic effect of the immunogenic composition can be tested by administering the composition to the test animal after implantation of cancer cells and establishment of a tumour and monitoring the growth and/or metastasis of the tumour.

### Uses

The present invention provides for a number of uses for the immunogenic compositions and combination products comprising OmpC and/or OmpF. Non-limiting examples include the use of the immunogenic composition as an adjuvant or immunostimulant or, when combined with antigenic material, as a vaccine, including in cases where the immunogenic composition comprises antigenic material from more than one disease-causing organism, as a multivalent vaccine. Combination products can be used as vaccines, including, as above, in cases where the product comprises antigenic material from more than one disease-causing organism, as multivalent vaccines. The present invention thus also provides methods of potentiating an immune response to antigenic material in a subject comprising administering an effective amount of OmpC and/or OmpF in combination with the antigenic material. Also provided are methods of inducing an immune response in a subject by administering an immunogenic composition or combination product of the invention.

The products of the invention are suitable for use in humans as well as non-human animals, including domestic and farm animals. The administration regime for the product need not differ from any other generally accepted vaccination programs. For example, when the product is an immunogenic composition comprising OmpC and/or OmpF in combination with antigenic material, a single administration of the product in an amount sufficient to elicit an effective immune response may be used or, alternatively, other regimes of initial administration of the immunogenic composition followed by boosting with antigen alone or with the immunogenic composition may be used. Likewise, when the product is a combination product, the preparation comprising OmpC and/or OmpF may be combined with the antigenic material formulation (such as a commercial vaccine) and administered as a single composition, with the option of subsequent boosters with the OmpC and/or OmpF preparation alone, the antigenic material alone or a combination of the two. Alternatively, the OmpC and/or OmpF preparation may be administered separately from the antigenic material formulation. In this case, the OmpC and/or OmpF preparation may be administered prior to or subsequent to administration with the antigenic material formulation. Optional boosters of either the OmpC and/or OmpF preparation or the antigenic material formulation or both may also be included in the regime. Boosting in either administration regime may occur at times that take place well after the initial administration, for example, if antibody titres fall below acceptable levels. The exact mode of administration of the product will depend for example on the components of the composition, the subject to be treated and the desired end effect of the treatment. Appropriate modes of administration can be readily determined by the skilled practitioner.

The product can be used prophylactically, for example to prevent infection by a virus, bacteria or other infectious particle, or development of a tumour or other disease, or it may be used therapeutically to ameliorate the effects of a disease or disorder associated with an infection or of a cancer or other disease. In one embodiment of the invention, the product is used prophylactically.

The product can be used in the prevention or treatment of a variety of diseases or disorders depending on the antigenic material selected for inclusion in or use with the product. Non-limiting examples include various virally- or bacterially-related diseases, such as influenza (using antigenic material from various influenza viruses), typhoid fever (using antigenic material from S. typhi), HCV infections (using HCV antigenic material), HBV infections (using HBV antigenic material), HAV infections (using HAV antigenic material), HIV infections (using HIV antigenic material), polio (using poliovirus antigenic material), diptheria (using antigenic material derived from diptheria toxin), tuberculosis (using Mycobacterium tuberculosis antigenic material), EBV infections (using EBV antigenic material), as well as allergic reactions (using various allergens) and cancer (using various tumour-associated antigens). Other uses include, for example, prevention or treatment of inflammatory diseases (for example, arthritis); infections by avian flu virus, human respiratory syncytial virus, Dengue virus, measles virus, mumps virus, rubella virus, Varicella zoster virus, variola virus, herpes simplex virus, human papillomavirus, pseudorabies virus, swine rotavirus, swine parvovirus, Newcastle disease virus, foot and mouth disease virus, hog cholera virus, African swine fever virus, infectious bovine rhinotracheitis virus, infectious laryngotracheitis virus, La Crosse virus, neonatal calf diarrhea virus, bovine respiratory syncytial virus, bovine viral diarrhea virus, Mycoplasma hyopneumoniae, Streptococcal bacteria, Gonococcal bacteria, Enterobacteria or parasites (for example, leishmania or malaria).

In one embodiment of the invention, there is provided an immunogenic composition or combination product comprising OmpC and/or OmpF and antigenic material derived from the influenza virus and the use of the immunogenic composition or combination product as a vaccine against influenza. In one embodiment of the invention, there is provided an immunogenic composition or combination product comprising OmpC and optionally OmpF and antigenic material derived from the influenza virus and the use of the immunogenic composition or combination product as a vaccine against influenza. In another embodiment, there is provided a use of an immunogenic composition of the invention comprising OmpC and optionally OmpF to adjuvant the immunoprotective effect of a pre-formulated influenza vaccine.

In another embodiment, the invention provides for an immunogenic composition or combination product comprising OmpC and/or OmpF and antigenic material derived from the influenza virus capable of providing protection against a plurality of influenza virus strains. In a specific embodiment, the antigenic material included in to for use with the products of the invention is in the form of a pre-formulated influenza vaccine and the porin(s) act to adjuvant the effects of the pre-formulated vaccine such that it provides protection against heterologous strains of influenza. In another embodiment, the invention provides for the use of OmpC and/or OmpF to adjuvant a pre-formulated influenza vaccine such that it provides protection against heterologous strains of influenza.

In one embodiment of the invention, the product comprises OmpC and optionally OmpF and a pre-formulated influenza vaccine and the ratio of OmpC to influenza vaccine ranges between about 2:1 and about 1:100 (by weight). In another embodiment, the product comprises OmpC and optionally OmpF and a pre-formulated influenza vaccine and the ratio of OmpC to influenza vaccine is between about 1:1 and about 1:20 by weight. In other embodiments, the product comprises OmpC and optionally OmpF and a pre-formulated influenza vaccine and the ratio of OmpC to influenza vaccine is between about 1:2 and about 1:20 by weight, for example, between about 1:2 and about 1:15 by weight; between about 1:3 and about 1:15 by weight; between about 1:4 and about 1:15 by weight, or between about 1:5 to about 1:15 by weight. In another embodiment, the product comprises OmpC and optionally OmpF and a pre-formulated influenza vaccine and the ratio of OmpC to influenza vaccine is between about 1:1 and about 1:10 by weight.

The present invention also provides for the use of the products as multivalent vaccine, either by including antigenic material from more than one disease-causing agent in the immunogenic composition or combination product, or by virtue of the ability of OmpC and OmpF themselves to provide protection against S. typhi infection. Additionally, due to the high homology between OmpC and OmpF of Salmonella spp. and the corresponding porins in other enterobacteria, in some embodiments, Salmonella spp. OmpC and/or OmpF can also be used to provide protective effects against infection with other enterobacteria.

In one embodiment, there is provided the use of a product of the invention comprising OmpC and/or OmpF and antigenic material from more than one disease-causing organism as a multivalent vaccine. Examples of combinations of antigenic material suitable for use in the multivalent vaccines of the invention include, but are not limited to, antigenic material from hepatitis A and hepatitis B viruses to provide a multivalent vaccine against hepatitis A/hepatitis B; antigenic material from hepatitis B virus and H. influenzae type b to provide a multivalent vaccine against hepatitis B/Hib; antigenic material from Corynebacterium diphtheriae and Clostridium tetani to provide a multivalent vaccine against tetanus and dipheria; antigenic material from Corynebacterium diphtheriae, Clostridium tetani and Bordetella pertussis to provide a multivalent vaccine against diphtheria/tetanus/pertussis; antigenic material from Corynebacterium diphtheriae, Clostridium tetani, Bordetella pertussis and H. influenzae type b to provide a multivalent vaccine against diphtheria/tetanus/pertussis/Hib; antigenic material from Corynebacterium diphtheriae, Clostridium tetani, Bordetella pertussis, polio virus and hepatitis B virus to provide a multivalent vaccine against diphtheria/tetanus/pertussis/polio/hepatitis B; antigenic material from the mumps virus, measles virus and rubella virus to provide a multivalent vaccine against measles/mumps/rubella (MMR), and antigenic material from the mumps virus, measles virus, rubella virus and varicella zoster virus to provide a multivalent vaccine against MMR/chickenpox.

In one embodiment of the invention, there is provided a products of the invention comprising S. typhi OmpC and/or OmpF and antigenic material from one or more other disease-causing agent for use to provide protection against S. typhi infection and protection against the one or more other disease-causing agent. In another embodiment, there is provided a use of the products of the invention to provide protection against S. typhi infection and protection against one other disease-causing agent. Examples of combinations of antigenic material for inclusion in the products of the invention include, but are not limited to, the combinations listed above. As is known in the art, typhoid fever is most prevalent in third world and/or tropical countries, as are a number of other diseases, such as, amoebic dysentery (amoebiasis), shigellosis, cholera, meningococcal meningitis, yellow fever, Dengue fever, encephalitis, West Nile virus disease, hepatitis, malaria, rotavirus infections, human papilloma virus infections, Chlamydia infections, SARS infections, Vancomycin-resistant S. aureus infections, Cryptosporidiosis, Hanta virus infections, Epstein Barr virus infections, Cytomegalovirus infections, H5N1 Influenza, Enterovirus 71 infections, E. coli O157:H7 infections, Human Monkey pox, Lyme disease, Cyclosporiasis, Hendra virus infections, Nipah virus infections, Rift Valley fever, Plague, Marburg haemorrhagic fever, Whitewater arrollo virus infections and the like. One embodiment of the invention provides for the use of the products of the invention to provide protection against S. typhi infection and protection against one or more of the causative agents of amoebic dysentery, shigellosis, cholera, meningococcal meningitis, yellow fever, Dengue fever, encephalitis, West Nile virus disease, hepatitis, or malaria. Such multivalent vaccines are not only useful for individuals who live in countries where such diseases are prevalent, but also for travellers planning to visit countries where these diseases are prevalent.

In a specific embodiment, there is provided a product of the invention comprising S. typhi OmpC and/or OmpF and antigenic material derived from the influenza virus and the use of this product to provide protection against typhoid fever and influenza. In another embodiment, there is provided a combination product comprising OmpC and optionally OmpF in combination with a commercial influenza vaccine, for use to provide protection against typhoid fever and influenza.

In one embodiment of the invention, there is provided a combination product comprising OmpC and/or OmpF and a vaccine comprising multiple epitopes, for example, a whole cell vaccine (such as an attenuated or inactivated viral vaccine, or complex mixture of proteins and/or other cellular components, such as a subunit vaccine). In another embodiment of the invention, an immunogenic composition comprising OmpC and/or OmpF is used to potentiate protective effects against diseases or disorders that require the efficient induction of B and T cell responses in order to be effective, such as influenza, hepatitis B, hepatitis C, HIV infections, human T-lymphotropic virus (HTLV) infections, Dengue virus infection, malaria, and systemic bacterial infections (such as those that occur in typhoid fever, Leishmania major infection and Mycobacterium tuberculosis infection).

In one embodiment, the products according to the invention are capable of providing a long-lasting immune response that confers protection on the vaccinated subject for a period of several months after vaccination. In accordance with this embodiment of the invention, therefore, the product is used prophylactically to provide a long-lasting immune response capable of protecting the vaccinated subject for a period of several months, for example, between about 2 months and about 10 months, after vaccination. In a specific embodiment in which the product comprises antigenic material from influenza virus, the product is used to provide protection in a subject against infection with an influenza virus for 6 months or more, for example, at least 7, at least 8, at least 9, or at least 10 months after vaccination.

One embodiment of the present invention also provides for the use of OmpC and/or OmpF to adjuvant DNA vaccines.

### Kits

The present invention additionally provides for kits comprising the immunogenic compositions or combination products. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the product.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Purification of Salmonella typhi Porin Proteins

The following purification procedure was used for purification of the porins OmpC and OmpF from S. typhi. The purification procedure is based on that described by Secundino et al. (2006), Immunology 117:59.

The two proteins were co-purified from Salmonella typhi. Individual purification of OmpC and OmpF was achieved using knock-out mutants of S. typhi in which either OmpC [STYC171 (OmpC⁻)] or OmpF [STYF302 (OmpF⁻)] open reading frames have been interrupted. The procedure for purification of the individual porins from the knock-out mutated forms of the bacteria was followed as for the co-purification. This procedure is outlined below.

The bacterial strain, Salmonella typhi 9,12,Vi:d (ATCC 9993) was grown in Minimal medium A supplemented with yeast extract, magnesium and glucose at 37° C., 200 rpm. The formula for 10 L Minimal medium A supplemented with yeast extract, magnesium and glucose is: 5.0 g of dehydrated Na-Citrate (NaC₆H₅O₇:2H₂O), 31.0 g NaPO₄ monobasic (NaH₂PO₄), 70.0 g NaPO₄ dibasic (Na₂HPO₄), 10.0 g (NH₄)₂SO₄, 200 mL yeast extract solution 5% (15.0 g in 300 mL). 1.434 L medium was distributed per 4 L Erlenmeyer flask. Sterilization was performed at 121° C., 15 lbs pression/in², 15 min. To each flask was then added: 6.0 mL of a 25% (w/v) sterile MgSO₄ solution and 60.0 mL of a 12.5% (w/v) glucose solution. The flask was inoculated with an overnight culture of S. typhi and when the OD₅₄₀ reached 1.0, incubation was stopped and the culture centrifuged at 7,500 rpm for 15 min at 4° C. The pellet was resuspended in 100 mL final volume of Tris-HCl pH 7.7 (6.0 g Tris-base/L) and the biomass was sonicated for 90 min on ice and then centrifuged at 7,500 rpm for 20 min at 4° C. To each 10 mL of supernatant was added: 2.77 mL 1M MgCl₂, 25 ml RNaseA (10,000 U/mL) and 25 ml DNaseA (10,000 U/mL). The mixture was then incubated at 37° C., 120 rpm for 30 min.

Porin extraction from the mixture was performed as follows:

The mixture was ultracentrifuged at 45,000 rpm, 4° C. for 45 min, and the pellet retained. The pellet was resuspended in 10 mL 5 mL Tris-HCl containing 2% (w/v) SDS and then homogenised. The homogenised mixture was incubated at 32° C., 120 rpm for 30 min. The incubated mixture was ultracentrifuged at 40,000 rpm, 20° C. for 30 min, and the pellet retained. The pellet was resuspended in 5 mL Tris-HCl containing 2% (w/v) SDS and then homogenised. The homogenized pellet was incubated at 32° C., 120 rpm for 30 min. The incubated mixture was ultracentrifuged at 40,000 rpm, 20° C. for 30 min, and the pellet retained. The pellet was resuspended in 20 mL Nikaido buffer containing 1% (w/v) SDS and then homogenised. [For 1 L of Nikaido buffer containing 1% (w/v) SDS: 6.0 g Tris-base, 10.0 g SDS, 23.4 g NaCl, 1.9 g EDTA was dissolved in water and the pH adjusted to pH 7.7. 0.5 mL [3-mercaptoethanol solution was then added]. The mixture was incubated at 37° C., 120 rpm for 120 min. The incubated mixture was ultracentrifuged at 40,000 rpm, 20° C. for 45 min. The supernatant, which contained the porin extract, was recovered.

The porins were purified from the supernatant using fast protein liquid chromatography (FPLC). 0.5× Nikaido buffer (see above) without 3-mercaptoethanol was employed during the purification process. The proteins were separated using a Sephacryl S-200 (1-PLC WATERS 650 E) with a Flux speed of 10 mL/min. The column was loaded with 22 mL of supernatant. Eluted fractions were monitored at 260 nm and 280 nm. The main peak, which contained the purified porins, was retained and stored at 4° C. The purified porins were stable for long period (over one year).

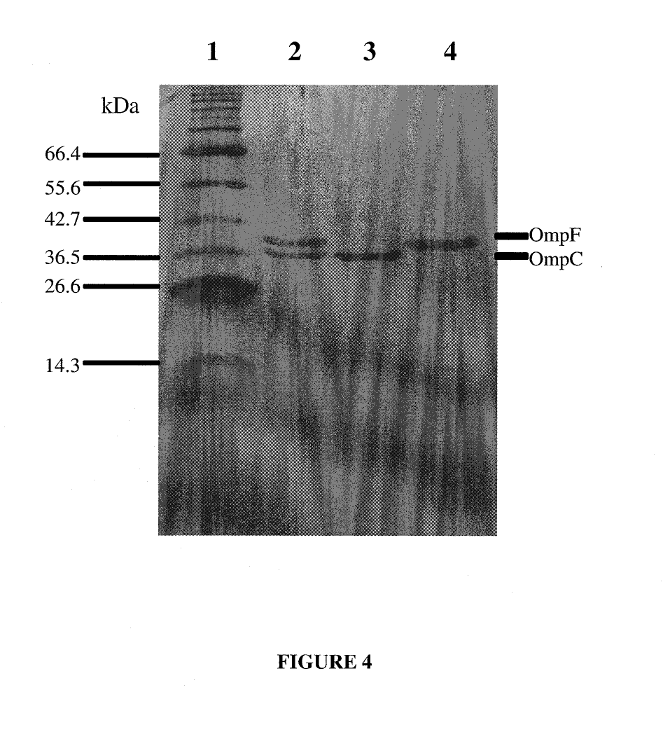

FIG. 4 shows the SDS-PAGE profile of the porins, OmpC and OmpF, purified by the procedure described above.

### Example 2

### S. typhi Porins Efficiently Induce Recruitment of Innate and Adaptive Immunity Cell Populations at the Immunization Site

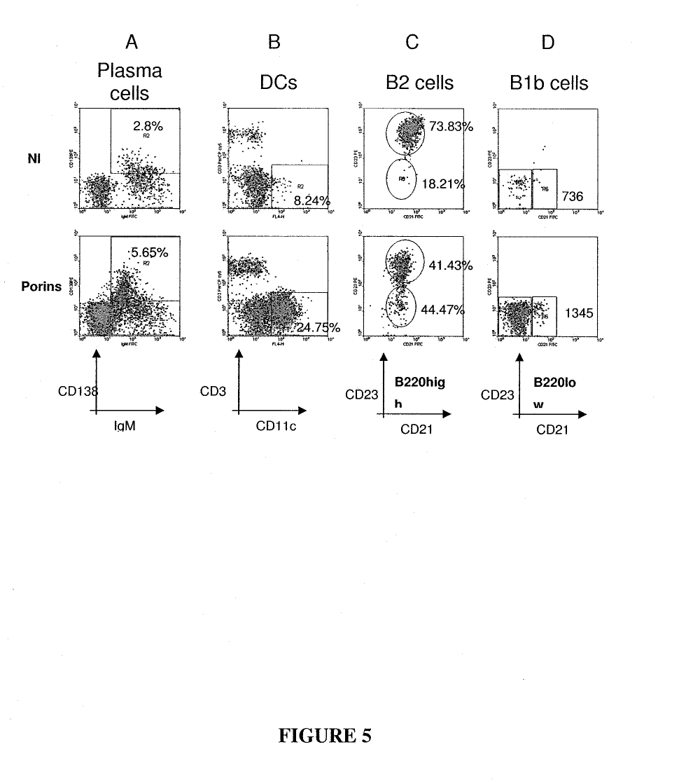

A mechanism proposed for adjuvant potentiation of immune responses is the recruitment of innate and adaptive immune cells to efficiently capture antigen avoiding its diffusion and depuration by other physiological mechanisms. In addition, these cells would promote antigen processing and presentation to activate adaptive immunity. In order to characterize the cell populations induced by immunization with S. typhi porins, BALB/c mice were immunized i.p. with 20 μg of porins (prepared as described in Example 1). 4 days after immunization, peritoneal exudate cells were obtained and stained for T cells (CD3+/CD4+), macrophages (CD11b+CD11c−/B220−), Plasma cells (CD138+IgMlow), Dendritic cells (CD11c+), B2 cells (CD21low/CD23low) and B1b cells (B220 low/CD5−/CD21−/CD23−) and subjected to FACS analysis. The percentage amounts of T cells and Macrophages were not significantly modified after immunization with the porins, however, an increase in the percentage of Plasma cells, DC and B2 cells was observed, as was an increase of B1b cell numbers (FIG. 5). These data show that immunization with S. typhi porins efficiently induces the recruitment of important innate and adaptive immunity cell populations at the site of injection. No signs of immunopathology or acute inflammation responses were observed indicating that non-pathological pro-inflammatory response was generated.

### Example 3

### S. typhi Porins Up-Regulate the Expression of Co-stimulatory Molecules and Activation Markers in Antigen Presenting Cells (APC)

Macrophages and dendritic cells are considered central cell populations for innate recognition to trigger and orchestrate adaptive immune responses. These cells are important contributors to the adjuvant effect by inducing co-stimulation to T cells and secreting important cytokines for B and T cell responses. Since S. typhi porins induced recruitment of DC at the site of immunization (see Example 2), the capacity of these proteins to induce up-regulation of co-stimulatory molecules and activation markers in these cells as well as in other important APC such as macrophages was investigated.

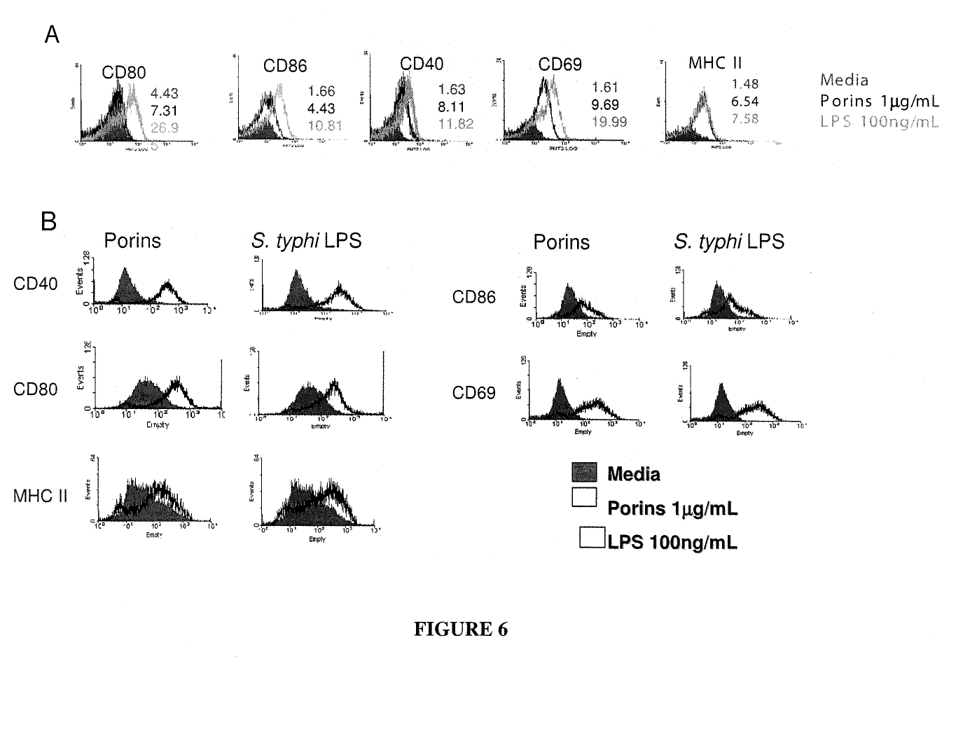

DC or Bone Marrow Derived Macrophages (BMDM) were stimulated with 1 μg/mL of S. typhi porins (prepared as described in Example 1) for 24 hours for DC and 48 hours for BMDM, then cells were stained with anti-CD80 anti-CD86, anti-CD40, anti-CD69, or anti-MHCII antibodies and flow-cytometry analysis was performed. Porins induced up-regulation of all the molecules tested (FIGS. 6A and 6B). The porin preparations did not show LPS contamination as measured by the standard Limulus test. The calculated detection limit of this test is 0.2 ng of LPS per microgram of porin.

The capacity of 0.2 ng of LPS to induce up-regulation of the molecules described above on DC and on BMDM was also tested. No up-regulation of these molecules was observed in these cells. Additionally, the capacity of a LPS preparation containing 100 ng of LPS (500 times the LPS concentration corresponding to the Limulus detection limit) to induce up-regulation of the molecules described above on DC and on BMDM was tested. Compared to the porins, 100 ng of LPS induced a greater up-regulation of CD80, CD86, CD40, CD69 on DC (FIG. 6A). MHC-II expression was equally up-regulated by LPS or porins on these cells (FIG. 6A). In contrast, LPS and porins induced similar up-regulation of co-stimulatory molecules on BMDM. These data suggest that porins are recognized by important APC such as DC and macrophages and that this recognition prepares these cells to efficiently present antigen to T cells.

### Example 4

### S. typhi Porins Induce Signalling Through TLR-2 and TLR-4 and Induce Production of Pro- and Anti-Inflammatory Cytokines on Dendritic Cells

Several microbial components are considered to be Pathogen Associated Molecular Patterns (PAMP). Recognition of PAMP in the host is mediated by Pattern Recognition Receptors (PRR), which are proteins present in the blood stream, tissues and on cell surface as well as intracellular locations. In particular APC are equipped with a variety of these receptors. Among PRR, Toll Like Receptors (TLR) are an important group which mediate the recognition of multiple PAMP and adjuvants.

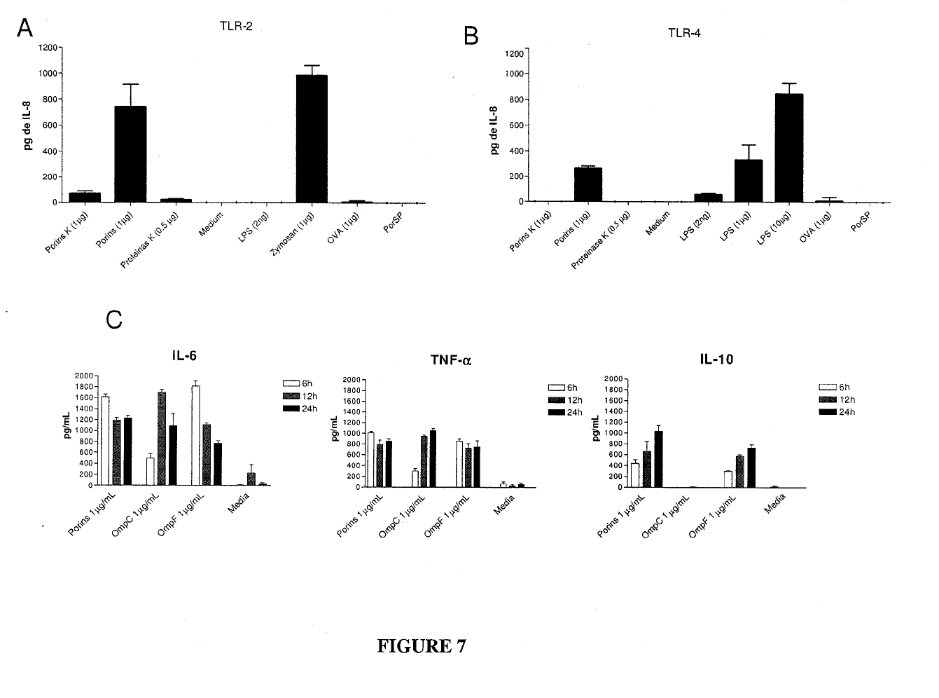

To test if S. typhi porins are a PAMP recognized by TLR, HEK 293 cells (which lack expression of TLR) transfected with plasmid encoding for TLR-4/MD2 (FIG. 7A) or TLR-2 (FIG. 7B) were stimulated with 1 μg/mL of S. typhi porins (prepared as described in Example 1), or with proteinase K degraded porins (Porins K), LPS, Zymosan (a glycan that binds to TLR-2) or a porin purification preparation in which S. typhi porins were depleted by flocculation/filtration (porins SP). TLR signaling was measured by reporter gene induction of IL-8 secretion using ELISA.

The S. typhi porin preparation efficiently induced IL-8 secretion on TLR-2 and TLR-4 transfected cells. IL-8 secretion was lost when porins were degraded (Porins K) indicating that besides porins no other TLR-2 or TLR-4 ligands contributed to the signal observed. 0.2 ng of LPS induced one-fifth of the amount IL-8 induced by porin stimulation indicating that the possible traces of LPS not detected by the Limulus test in the porin preparation do not significantly contribute to the response observed. A porin preparation depleted of porins by flocculation/filtration (Porins SP) was used to determine whether any undetected PAMP contamination was present in the S. typhi porin preparation. Porins SP did not induce IL-8 secretion indicating that any potential PAMP contamination in the S. typhi porin preparation did not contribute to the observed signal. LPS-free ovalbumin (OVA) was used to control any protein-induced false signals in the system. The observation that S. typhi porins signal through TLR-2 and TLR-4 indicates that the porins are PAMP and further indicates that these proteins have intrinsic adjuvant properties.

Among several cellular effects induced by TLR engagement, the secretion of cytokines is of particular relevance for adjuvant effects. To investigate the effects of S. typhi porins on cytokine production on APC, DC were stimulated with 1 μg/mL of the S. typhi porin preparation, as well as OmpC and OmpF individually (all prepared as described in Example 1). Six, 12 or 24 hours after stimulation, culture supernatants were collected and the presence of IL-6, TNF-α and IL-10 was analyzed by ELISA (FIG. 7C). All preparations efficiently induced the secretion of IL-6 and TNF-α. The porin preparation and OmpF, but not OmpC, also induced IL-10 secretion. These data suggest that the intrinsic adjuvant properties of the porins could be mediated by efficient induction of pro-inflammatory responses. The data suggest that OmpC has superior adjuvant properties compared to the porin preparation and OmpF.

### Example 5

### S. typhi Porins Induce a Long-Lasting Adjuvant Effect on the Antibody Response to Model Antigens

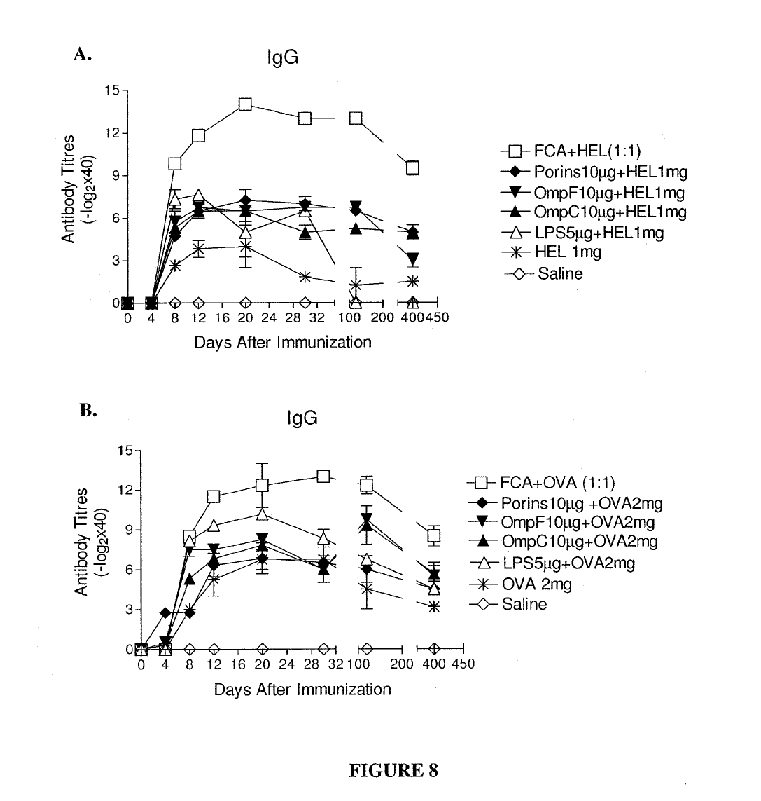

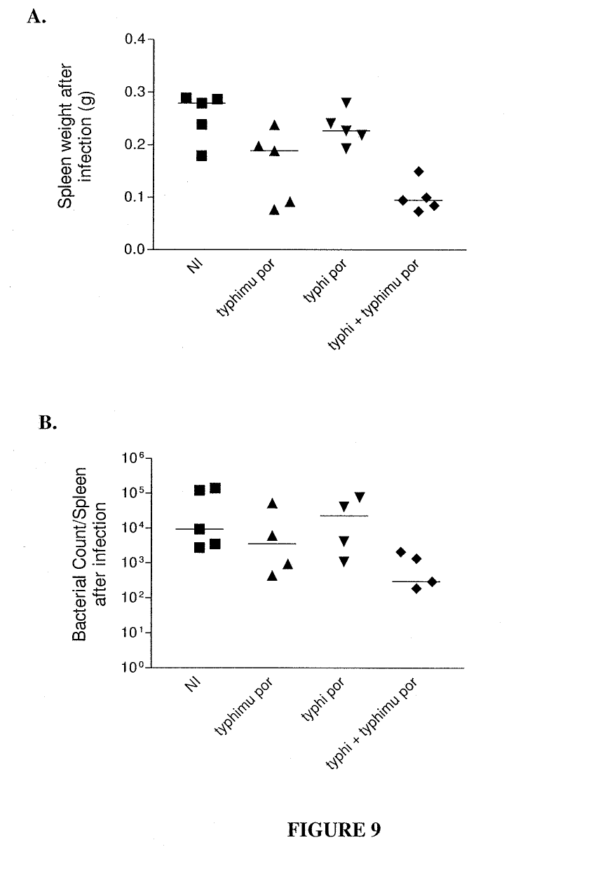

To test the adjuvant properties of the S. typhi porin preparation, groups of 3 BALB/c mice were co-immunized with 10 μg of either the porin preparation, OmpC or OmpF (all prepared as described in Example 1) mixed with 1 mg of hen egg lysozyme (HEL) (FIG. 8A) or 2 mg of OVA (FIG. 8B). 5 μg of LPS and Freund's Complete Adjuvant (FCA) (both recognized adjuvants) were used as controls. Specific IgG titers on the mice sera were measured by ELISA. The porin preparation, as well as OmpC and OmpF individually, all induced higher specific antibodies compared to HEL alone. These titers were similar to the titers induced by the co-immunization of HEL with LPS (FIG. 8A). Around day 100 post-immunization, however, HEL-specific antibody titers in the LPS group dropped to undetectable levels, whereas in the porin groups, HEL-specific titers remained until day 400 post-immunization (the last point tested). An adjuvant effect was also observed in OVA-specific IgG antibody responses when OVA was co-immunized with the various porin preparation. The effect was most evident in the long-lasting response after day 100 post-immunization (FIG. 8B). In the first 30 days, LPS induced higher OVA-specific antibody titers compared to the porin preparations, however, on day 100 and 400 post-immunization, the porin preparations showed a better adjuvant effect (FIG. 8B). The long-lasting OVA-specific antibody titers induced by porin co-immunization indicate that the B-cell memory compartment was efficiently established. In addition, the presence of IgG indicated that appropriate cytokines and a co-stimulatory environment were generated and that the T-cell compartment was activated. FCA induced the strongest adjuvant effect, however, the complexity of this material is much higher than that of either the porin preparations or LPS (FCA is comprises paraffin oil, Arlacel A and Mycobacterium smegmatis). FCA belongs to a group of adjuvants with restricted use due to its strong undesired side effects.

### Example 6

### Co-Immunization of S. typhi Porins with an Experimental Vaccine Against S. typhimurium Induced an Increase in Protective Capacity

The main objective for vaccination is the establishment of long-term protective antibody and T cell responses leading to long-term immunity to infection. Since efficient induction of T and B cell responses to a given antigen do not always correlate to protection, the ability of the efficient adjuvant effect elicited by S. typhi porins on the immune response to model experimental antigens, as demonstrated in the previous Examples, to induce an increase in the protective capacity of an experimental vaccine against S. typhimurium was investigated. The experimental vaccine comprises S. typhimurium porins.

Groups of 5 C57/BL6 mice were immunized i.p. with 20 μg of S. typhi porins (typhi por), 20 μg of S. typhimurium porins (typhimu por) or both (typhi+typhimu por) on day 0. On day 35 mice were infected i.p. with 10⁴ CFU of PhoP⁻ S. typhimurium. Five days after infection (day 40), the spleen from each mouse was removed and the weight (FIG. 9A) and the number of bacteria per spleen (FIG. 9B) was determined. Protection correlates with the eradication of bacteria from the spleen and with the reduction in spleen weight since these parameters indicate a reduction in the inflammatory process that could lead to a systemic inflammatory response. Compared to non-immunized animals, mice vaccinated with S. typhimurium porins showed a 30% reduction of spleen weight (FIG. 9A) and close to a log₁₀ reduction in bacterial count. Immunization with S. typhi porins resulted in a reduction of approximately 15% on spleen weight but an increase in bacterial count was observed (FIG. 9B). Co-administration of S. typhi and S. typhimurium porins induced a 60% reduction on spleen weight (FIG. 9A) and a reduction of almost 2 log₁₀ in bacterial count (FIG. 9B). These data demonstrate that S. typhi porins efficiently promoted an increase in the protection capacity of the vaccine.

### Example 7

### Adjuvant Effect of OmpC on a Commercial Influenza Vaccine Fluviral®

The Fluviral® vaccine used in this Example is a commercially available trivalent, inactivated split-virion vaccine prepared in eggs (ID Biomedical Corporation, Date of Approval: May 2, 2007, GlaxoSmithKline Biologicals North America, Quebec City, QC, Canada) and comprises the influenza strains: A/Solomon Islands/3/2006, A/Wisconsin/67/2005, B/Malaysia/2506/2004.

BALB/c mice were divided into 3 groups, 5 per group, and immunised once via the subcutaneous route as follows:
Group I: 3 μg (equivalent to one-fifth the human dose) of the commercial influenza vaccine Fluviral®.
Group II: 3 μg (equivalent to one-fifth the human dose) Fluviral® with 3 μg of purified OmpC.
Group III: 3 μg (equivalent to one-fifth the human dose) Fluviral® with 30 μg of purified OmpC.

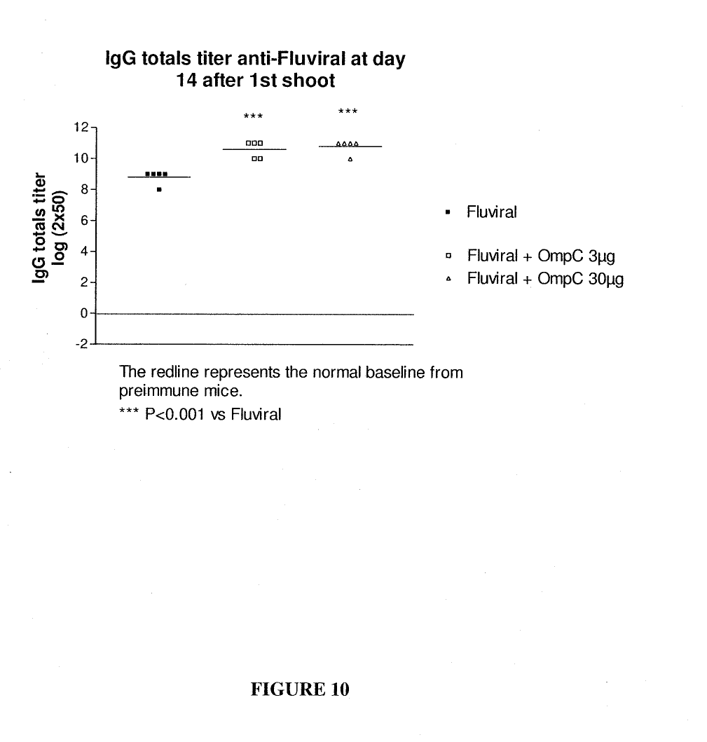

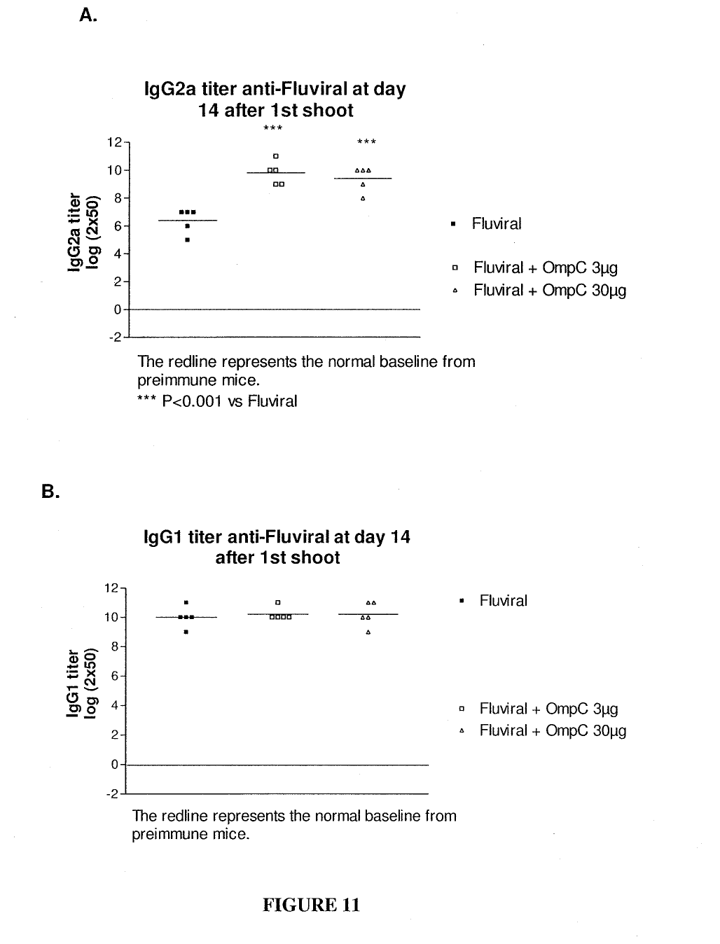

Blood was taken from the treated mice 14 days after injection and the total IgG was measured by ELISA toward the total Fluviral® proteins using peroxidase-conjugated goat anti-mouse IgG as secondary antibodies (Jackson ImmunoResearch Laboratories, Inc.). As can be seen from the results shown in FIG. 10, the addition of as little as 3 μg of OmpC improves the immune response to the Fluviral® vaccine by 4-fold after only one injection.

The levels of IgG2a directed to the Fluviral® proteins were also measured by ELISA. The IgG class switch that induces the production of IgG2a is indicative of the stimulation of a TH1 response in mice. As such, the presence of IgG2a suggests the triggering of a CTL response. As shown in FIG. 11A, both adjuvant regimens (Group II and Group III mice) induced an amount of IgG2a that was increased by a factor of 8-fold over non-adjuvanted Fluviral®. This is a striking improvement of the adjuvanted regimens over Fluviral® alone.

The amount of IgG1 did not increase when OmpC was added to the Fluviral® vaccine (FIG. 11B). IgG1 is a marker for the TH2 response. As noted above, the improvement observed in the IgG2a profile suggests that OmpC triggers a TH1 response, which is consistent with the fact that the IgG1 levels are not influenced.

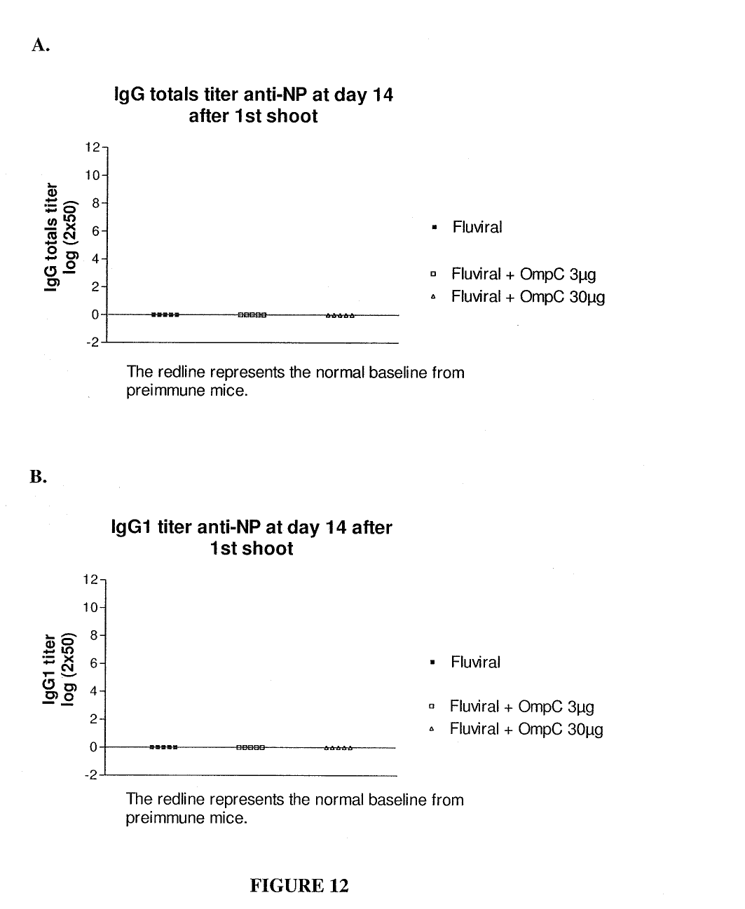

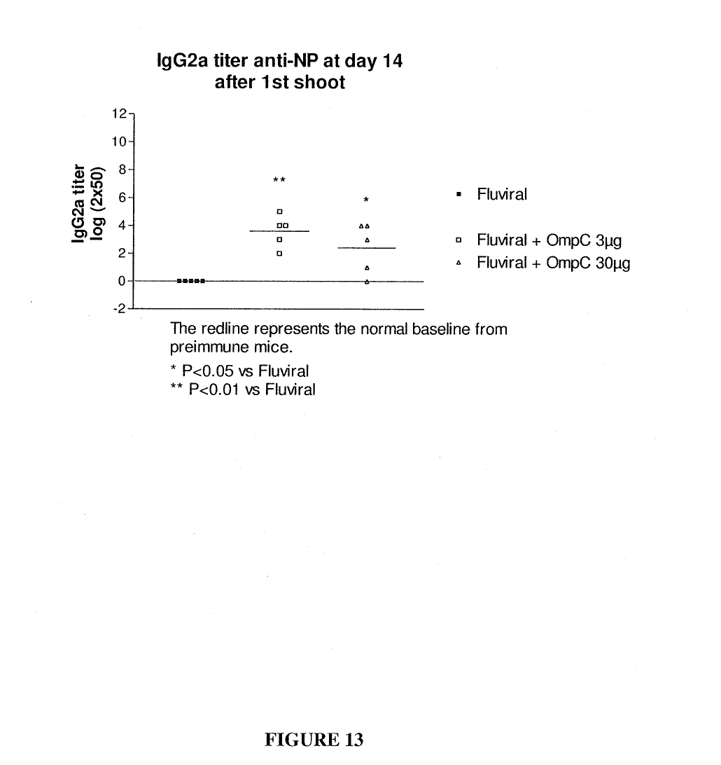

The NP protein is a conserved protein through all the strains of Influenza. However, as shown in FIGS. 12A & B, and FIG. 13, when the commercial Fluviral® vaccine is used to immunise mice, the immune response directed to this protein is negligible. Addition of either 3 μg or 30 μg OmpC to the Fluviral® vaccine, while not improving the IgG1 titers (FIG. 12B), improved the IgG2a titers to this highly conserved target considerably (FIG. 13). This result implies that a CTL response directed to NP was also triggered in those mice immunized with the Fluviral® vaccine adjuvanted with OmpC.

### Example 8

### Challenge of OmpC-Fluviral® Vaccinated Mice with Heterologous Influenza Strain WSN/33

To demonstrate that OmpC was able to induce protection to a heterologous strain of influenza, the following experiment was conducted. “Heterologous” in this context indicates a strain of influenza against which the commercial Fluviral® vaccine does not induce protection in mice.

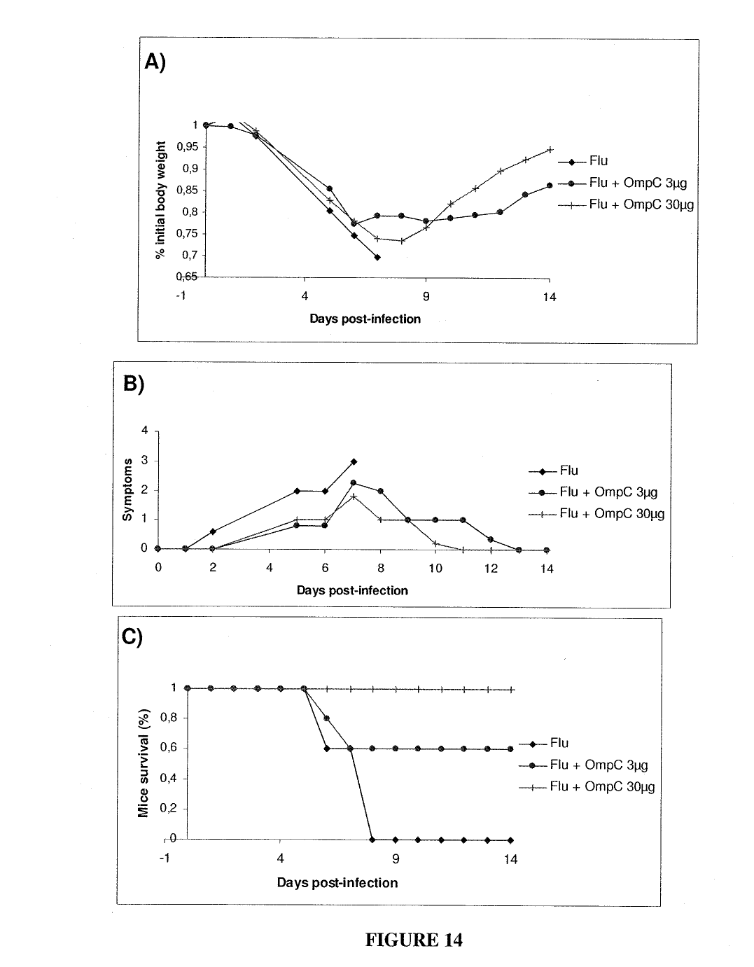

Mice vaccinated as described in Example 7 (Groups I-III) were challenged with 4,000 pfu (plaque forming units) of influenza strain WSN/33. All Group I mice (vaccinated with Fluviral® alone) were infected and showed a rapid decrease in body weight (FIG. 14A) and exhibited severe symptoms (FIG. 14B). The symptom legend for FIG. 14B is provided in Table 4 below. All Group I mice eventually died as a result of the infection (FIG. 14C).

In contrast, the mice vaccinated with Fluviral® adjuvanted with either 3 μg or 30 μg of OmpC lost less weight (FIG. 14A), showed less severe symptoms (FIG. 14B) and improved survival (FIG. 14C). While both groups of mice receiving the adjuvanted Fluviral® vaccine showed an improvement over those receiving the non-adjuvanted Fluviral® vaccine, the group receiving the higher dose of OmpC (30 μg) showed the best results with all mice surviving the infection. This result strongly suggests that the addition of OmpC to the Fluviral® vaccine triggered a CTL response in the mice toward highly conserved epitope of influenza, such as the NP protein. As a result, a complete protection to a lethal challenge of the heterologous WSN/33 strain of influenza was demonstrated.

TABLE 4
Symptoms legend for FIG. 14B

| Rating | Symptoms |
|---|---|
| 0 | No symptoms |
| 1 | Lightly spiked fur; Lightly |
| | curved back |
| 2 | Spiked fur; Curved back |
| 3 | Spiked fur; Curved back; |
| | Difficulty moving; Light |
| | dehydration |
| 4 | Spiked fur; Curved back; |
| | Difficulty moving; Severe |
| | dehydration/thin; Lifeless/ |
| | closed eyes; Ocular secretion |
| | This level leads to euthanasia |

The results shown in Examples 7 and 8 suggest that the addition of OmpC to commercially available influenza vaccines, such as the Fluviral® vaccine, will be sufficient to produce an influenza vaccine that will protect against a broad spectrum of influenza strains, including for example avian influenza. This protection is likely to be mediated at least in part through the induction of a CT response to conserved proteins of the influenza virus such as the NP protein.

### Example 9

### Adjuvant Effect of OmpC on the Commercial Influenza Vaccine Fluviral®

The Fluviral® vaccine used in this Example was the 2007-2008 version of the vaccine (as described in Example 7).

OmpC was produced and purified from the laboratory of Dr. C. Lopez Macias, IMSS, Mexico City. The protein was extracted from a mutant strain of Salmonella typhi (ΔOmpF) and was produced as described in Example 1 under GLP conditions.

BALB/c mice were divided into 2 groups, 10 mice per group, and immunised once at day 0 via the subcutaneous route at the back of the neck as follows:

Group I: 3 μg (equivalent to one-fifth the human dose) of the commercial influenza vaccine Fluviral®.

Group II: 3 μg (equivalent to one-fifth the human dose) of the commercial influenza vaccine Fluviral®, with 30 μg of purified OmpC.

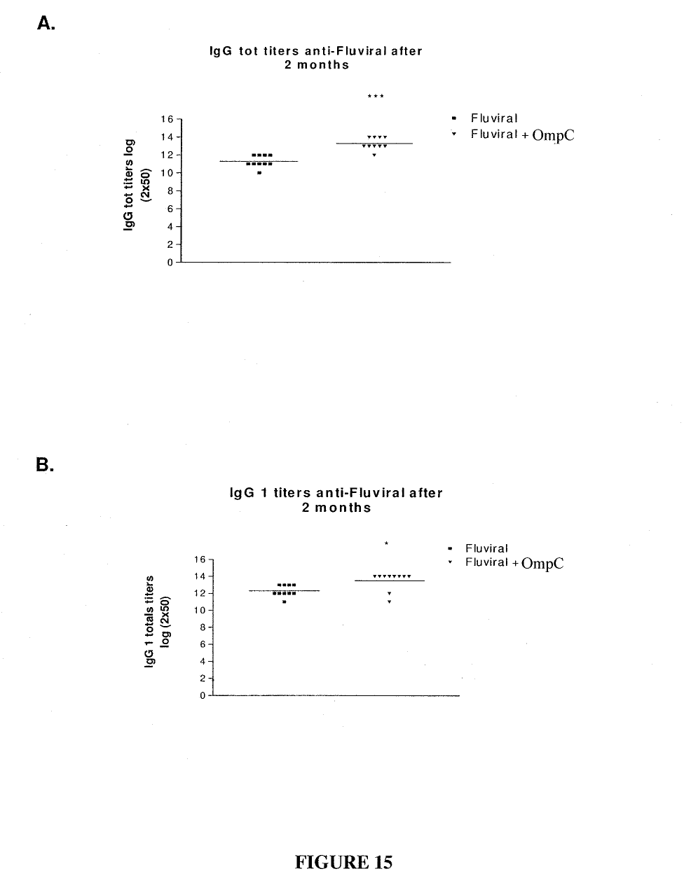

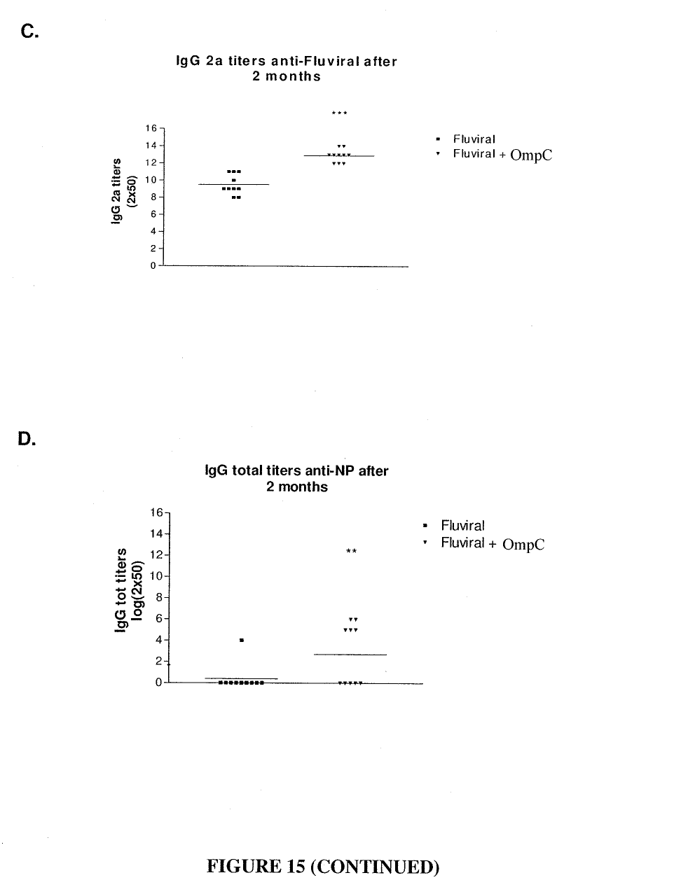

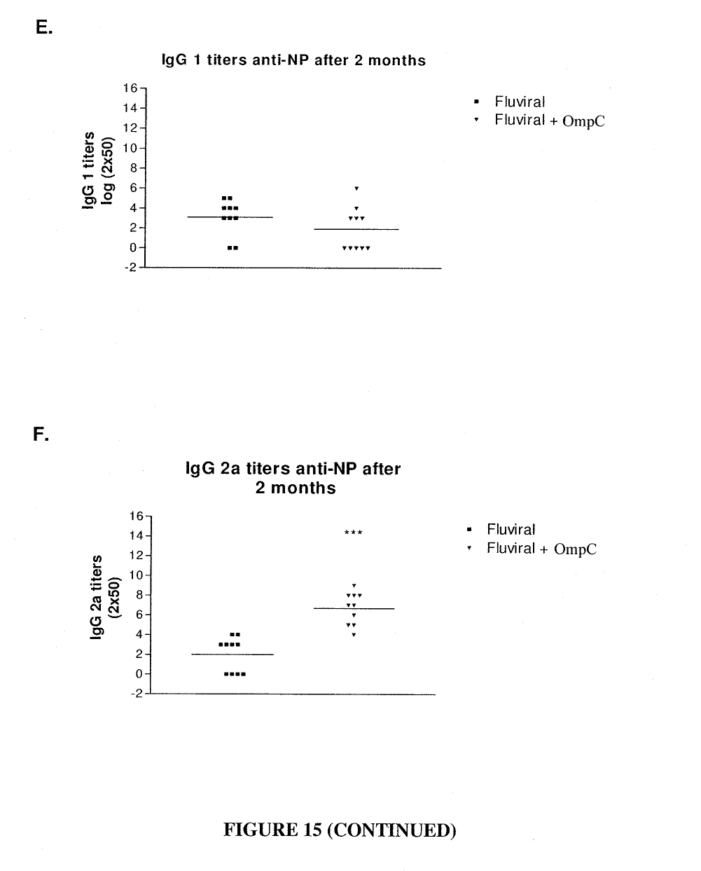

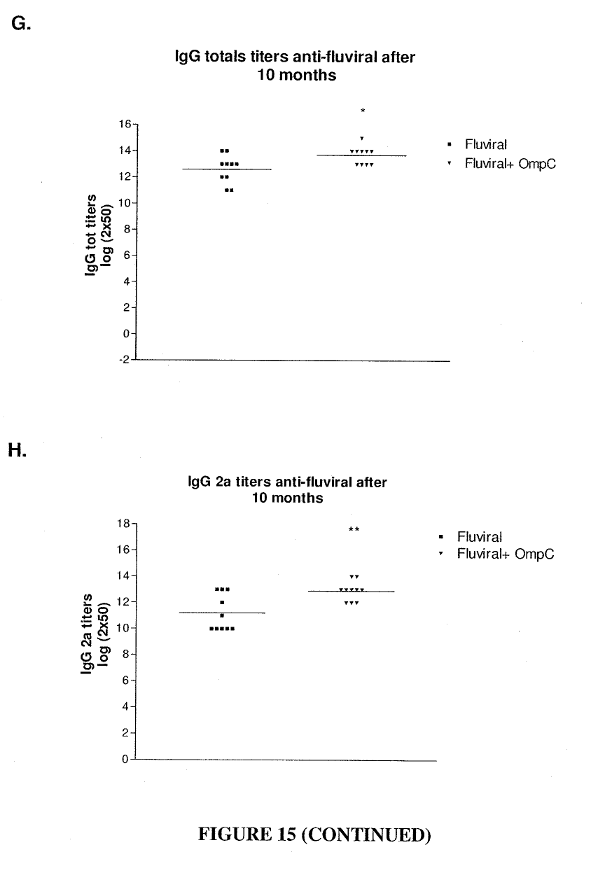

Blood samples were taken at day 0, and 8 weeks (2 months) and 40 weeks (10 months) after injection. The immune response directed against Fluviral proteins or against purified NP protein was measured as follows. ELISAs were performed on the blood samples taken at 2 months to measure the levels of total IgG, IgG1 or IgG2a raised against Fluviral® proteins or against the purified NP protein. The ELISAs were carried out as described in Example 7. The blood samples taken at 10 months were assayed to determine the levels of total IgG, and IgG2a directed against the Fluviral® proteins. The results are shown in FIG. 15, depicting total IgG (A), IgG1 (B) and IgG2a (C) to Fluviral® as measured 2 months after immunisation, total IgG (D), IgG1 (E) and IgG2a (F) to NP, also 2 months after immunisation, and total IgG (G), and IgG2a (H) against Fluviral® 10 months after immunization.

The results shown in FIG. 15 indicate that the addition of 30 μg of the OmpC adjuvant to the Fluviral® vaccine improved considerably the humoral response to the total Fluviral® proteins as showed by an increase of 4-fold of the total IgG directed to the vaccine (FIG. 15A). Furthermore, the results confirm that, as indicated in Example 7, the OmpC adjuvant triggers a bias toward the T_{H1} response since the increase of the IgG2a isotype when OmpC is used as an adjuvant is 32-fold higher than with Fluviral® alone (FIG. 15C). The IgG1 titers that are a marker for the T_{H2} response in this instance increased by 2-fold (FIG. 15B), but the increase was of a lesser magnitude than that for IgG2a. Taken together, these results suggest that OmpC is an adjuvant that increases both the T_{H1} and T_{H2} responses, but is more efficient in increasing the T_{H1} response. Also supportive of this conclusion are the results shown in FIG. 7, which show that OmpC can induce secretion of TNF-α (T_{H1}) and IL-6 (T_{H1}) cytokines. OmpF also induces secretion of these two cytokines as well as inducing in addition the secretion of IL-10, a T_{H2} cytokine.

The immune response to the highly conserved NP protein, which is present in the commercial Fluviral® vaccine, was also compared in mice vaccinated with the vaccine with and without OmpC as an adjuvant. As shown in FIG. 15, there was an increase of 4- to 8-fold in the total IgG titers (FIG. 15D) and 32-fold in the IgG2a titers (FIG. 15F) directed to this protein, indicating that OmpC is able to increase significantly the humoral response to this protein. This result is significant given that the NP protein is highly conserved amongst all strains of influenza.

Finally, the results indicate that the memory response induced by the adjuvant lasts for a long period (>10 months) as shown by the high levels of total IgG (FIG. 15G) and IgG2a (FIG. 15H) remaining toward the Fluviral® proteins in mice vaccinated with the combination of Fluviral® and OmpC.

### Example 10

### Challenge of OmpC-Fluviral® Vaccinated Mice with Heterologous Influenza Strain A/WSN/33

Similar to the experiment described in Example 8, the ability of OmpC to induce protection to a strain of influenza heterologous to those present in the Fluviral® vaccine was determined as follows.

The same two groups of mice immunized as described in Example 9 were challenged with 100 pfu (a dosage of ILD₅₀) of the influenza A/WSN/33 virus (intranasal administration in a volume of 500) at 10 months after immunization. After infection, the mice were monitored for weight loss, survival, and the development of symptoms (as outlined in Table 4).

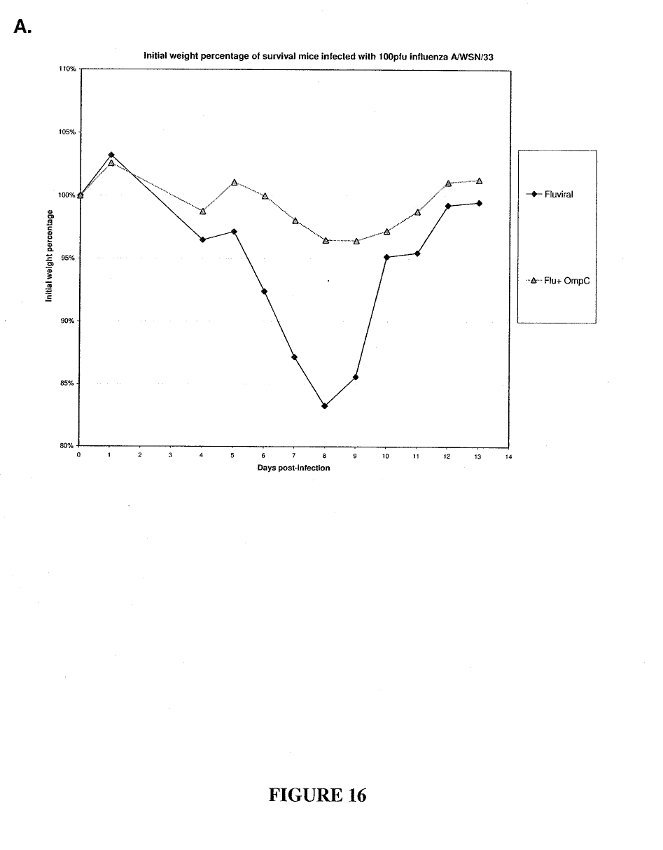

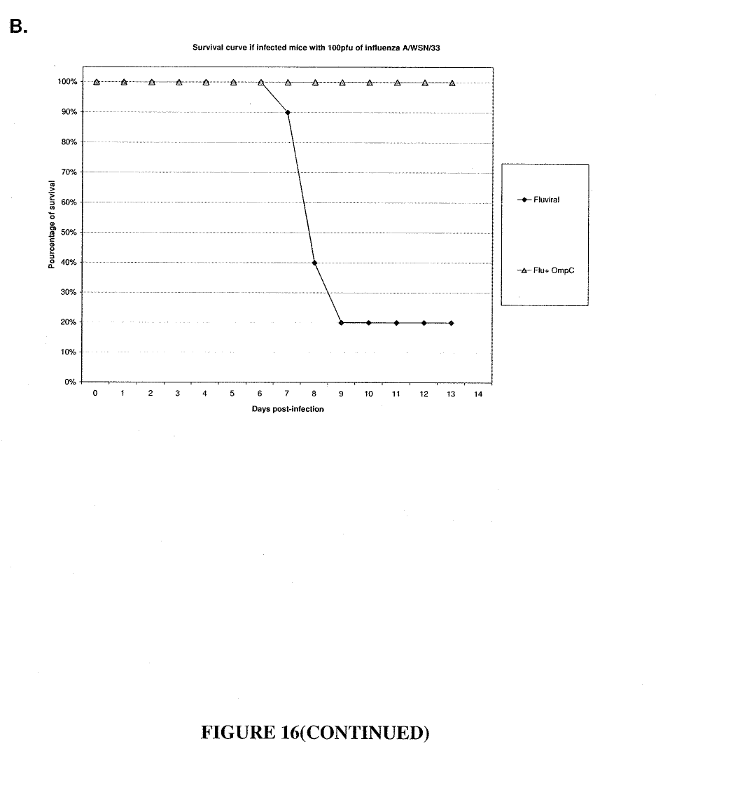

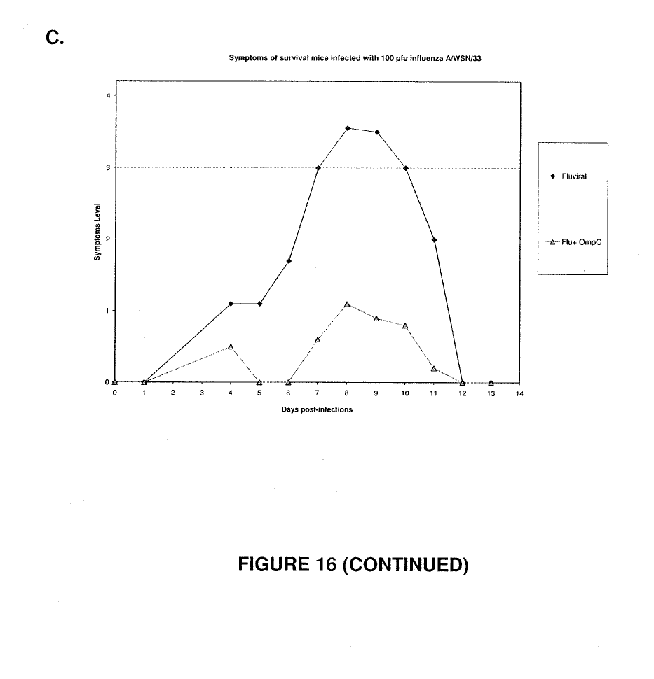

The results of this challenge are depicted in FIG. 16A to C, and indicate that mice vaccinated with Fluviral® alone were not protected from challenge with the heterologous influenza strain WSN/33. However, the addition of the adjuvant OmpC increased significantly the protection to infection as shown by a minimal weight loss (<5%) as compared to Fluviral® alone (>15%)(FIG. 16A), a survival of 100% (FIG. 16B) and development of very weak symptoms only (FIG. 16C).

The results described in Examples 9 and 10 suggest that the use of OmpC as an adjuvant when added to the Fluviral® vaccine is able to trigger a TH1 immune response to a highly conserved antigen like NP. The use of OmpC as an adjuvant as described also leads to the protection of mice vaccinated with the adjuvanted vaccine against a heterologous strain of influenza that is not found in the commercial Fluviral® vaccine. Therefore, from these results it is predicted that the addition of the adjuvant OmpC will enable the adjuvanted vaccine to protect against most, if not all, strains of influenza.

### Example 11

### OmpC Adjuvant Effect on the Antibody Response to a Mycobacterium tuberculosis Experimental Vaccine

An experimental vaccine against Mycobacterium tuberculosis containing the p38 protein (see Espitia C, et al. (1989) Clin Exp Immunol. September; 77(3):373-7; Espitia C, and Mancilla R. (1989) Clin Exp Immunol. September; 77(3):378-83; and Castañon-Arreola M, et al. (2005) Tuberculosis (Edinb). January-March; 85(1-2):115-26. Epub 2005 Jan. 22) was used in this Example.

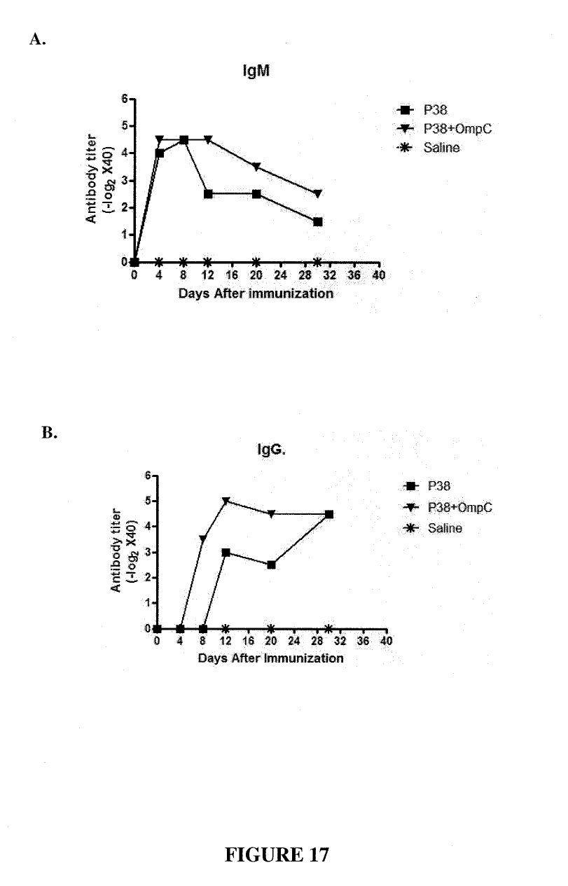

BALB/c mice were immunized intraperitoneally with either 10 μg of p38 protein or 10 μg of p38 protein in combination with 10 μg OmpC porin purified from S. typhi. Control mice were immunized with saline isotonic solution (saline) Blood samples were collected at various intervals as indicated in FIG. 17. Individual serum samples were maintained at −20° C. until analysis. Anti-p38 specific antibody titers (IgM and IgG) were measured by ELISA using standard protocols.

The results are shown in FIG. 17. While these results are of a preliminary nature, they are indicative of the ability of OmpC to adjuvant effectively this experimental Mycobacterium tuberculosis vaccine.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1-30. (canceled)

31. A method of potentiating an immune response in a subject, said method comprising administering to said subject an effective amount of an adjuvant and antigenic material, wherein said adjuvant comprises OmpC porin, or OmpF porin, or a combination thereof.

32. The method according to claim 31, wherein said OmpC porin has an amino acid sequence substantially identical to Salmonella typhi OmpC porin and said OmpF porin has an amino acid sequence substantially identical to Salmonella typhi OmpF porin.

33. The method according to claim 31, wherein said adjuvant comprises OmpC porin.

34. The method according to claim 31, wherein said adjuvant and said antigenic material are administered as a single formulation.

35. The method according to claim 31, wherein said adjuvant and said antigenic material are administered as separate formulations.

36. The method according to claim 31, wherein said antigenic material is a pre-formulated vaccine.

37. The method according to claim 31, wherein said antigenic material is derived from one or more strains of influenza virus.

38. The method according to claim 37, wherein said immune response comprises an immune response to a conserved influenza antigen.

39. The method according to claim 37, wherein said immune response comprises a CTL immune response to a conserved influenza antigen.

40. The method according to claim 37, wherein said immune response provides protection against a plurality of influenza strains.

41. The method according to claim 31, wherein said immune response comprises humoral and cellular immune responses.

42. The method according to claim 31, wherein said subject is a human.

43. A method of improving the efficacy of a vaccine comprising administering to a subject said vaccine and an adjuvant comprising OmpC porin, or OmpF porin, or a combination thereof, whereby the subject treated with said vaccine and said adjuvant shows an improved immune response over a subject treated with said vaccine alone.

44. The method according to claim 43, wherein said OmpC porin has an amino acid sequence substantially identical to Salmonella typhi OmpC porin and said OmpF porin has an amino acid sequence substantially identical to Salmonella typhi OmpF porin.

45. The method according to claim 43, wherein said adjuvant comprises OmpC porin.

46. The method according to claim 43, wherein said improved immune response comprises a cellular immune response.

47. The method according to claim 43, wherein said vaccine is an influenza vaccine.

48. The method according to claim 47, wherein said vaccine comprises antigenic material from one or more influenza A strains and antigenic material from one or more influenza B strains.

49. The method according to claim 47, wherein said influenza vaccine is an inactivated whole virion or split virion vaccine.

50. The method according to claim 47, wherein said influenza vaccine is a trivalent, split virion vaccine.

51. The method according to claim 47, wherein said improved immune response comprises an immune response to a conserved influenza antigen.

52. The method according to claim 47, wherein said improved immune response comprises a CTL immune response to a conserved influenza antigen.

53. The method according to claim 47, wherein said improved immune response confers protection against one or more heterologous strains of influenza.

54. The method according to claim 43, wherein said subject is a human.

55. A product comprising OmpC porin, or OmpF porin, or a combination thereof, and antigenic material, wherein said OmpC porin, or OmpF porin, or combination thereof, is capable of potentiating an immune response to said antigenic material in a subject.

56. The product according to claim 55, wherein said OmpC porin has an amino acid sequence substantially identical to Salmonella typhi OmpC porin and said OmpF porin has an amino acid sequence substantially identical to Salmonella typhi OmpF porin.

57. The product according to claim 55, wherein said product comprises OmpC porin.

58. The product according to claim 55, wherein said improved immune response comprises a cellular immune response.

59. The product according to claim 55, wherein said antigenic material is provided in the form of a pre-formulated vaccine.

60. The product according to claim 55, wherein said antigenic material is derived from one or more strains of influenza virus.

61. The product according to claim 60, wherein said antigenic material derived from one or more strains of influenza virus is in the form of a pre-formulated influenza vaccine.

62. The product according to claim 60, wherein said antigenic material derived from one or more strains of influenza virus includes antigenic material from one or more influenza A strains and antigenic material from one or more influenza B strains.

63. The product according to claim 61, wherein said pre-formulated influenza vaccine is an inactivated whole virion or split virion vaccine.

64. The product according to claim 61, wherein said pre-formulated influenza vaccine is a trivalent, split virion vaccine.

65. (canceled)

66. The method according to claim 31, wherein said antigenic material is a pre-formulated influenza vaccine.

67. The method according to claim 66, wherein said immune response comprises an immune response to a conserved influenza antigen.

68. The method according to claim 66, wherein said immune response comprises a CTL immune response to a conserved influenza antigen.

69. The method according to claim 66, wherein said immune response provides protection against a plurality of influenza strains.

70. The method according to claim 33, wherein said antigenic material is derived from one or more strains of influenza virus.

71. The method according to claim 70, wherein said antigenic material is in the form of a pre-formulated influenza vaccine.

72. The method according to claim 70, wherein said immune response comprises an immune response to a conserved influenza antigen.

73. The method according to claim 70, wherein said immune response comprises a CTL immune response to a conserved influenza antigen.

74. The method according to claim 70, wherein said immune response provides protection against a plurality of influenza strains.

75. The method according to claim 45, wherein said vaccine is an influenza vaccine.

76. The method according to claim 75, wherein said improved immune response comprises an immune response to a conserved influenza antigen.

77. The method according to claim 75, wherein said improved immune response comprises a CTL immune response to a conserved influenza antigen.

78. The method according to claim 75, wherein said improved immune response confers protection against one or more heterologous strains of influenza.

79. The product according to claim 57, wherein said antigenic material is derived from one or more strains of influenza virus.

80. The product according to claim 79, wherein said antigenic material derived from one or more strains of influenza virus is in the form of a pre-formulated influenza vaccine.
